(19) United States
(12) Patent Application Publication
(10) Pub. No.: US 2018/0333092 A1
(43) Pub. Date: Nov. 22, 2018

(54) PORTABLE OCULAR RESPONSE TESTING DEVICE AND METHODS OF USE

(71) Applicant: OPHTHALIGHT DIGITAL SOLUTIONS INC., Vancouver (CA)

(72) Inventors: Yaser Mohammadian Roshan, Vancouver (CA); Amirhossein Vejdani, Vancouver (CA); Farzad Hamidi, Vancouver (CA); Ehsan Daneshi Kohan, Vancouver (CA); Younes Rashidi, Vancouver (CA)

(21) Appl. No.: 15/777,285

(22) PCT Filed: Dec. 5, 2016
(86) PCT No.: PCT/CA2016/051426

§ 371 Date: May 18, 2018

Related U.S. Application Data
(60) Provisional application No. 62/262,581, filed on Dec. 3, 2015.
(60) Provisional application No. 62/369,995, filed on Aug. 2, 2016.

(51) Int. Cl. A61B 5/18; A61B 3/00; A61B 3/113; A61B 3/14; A61B 5/00; G06K 9/00; A61B 3/18

(52) U.S. Cl. CPC A61B 5/18; A61B 3/0008; A61B 3/0058; A61B 3/0091; A61B 3/113; A61B 5/4845; A61B 5/4863; G06K 9/0061; A61B 3/18; A61B 5/746; A61B 3/145

(57) ABSTRACT

A portable ocular response testing device includes a portable housing, a plurality of lights for generating visible light within two eye spaces of the portable housing, a controller for operating the lights according to a prescribed pattern, and an imaging system to capture images of the eyes under varying lighting conditions to evaluate an ocular response of a user to the lighting conditions. In some instances, the portable device is used in the field of ophthalmology for complete assessment of optic nerve defects producing Marcus-Gunn pupil, detection and quantification of color blindness, and detection and follow up of eye abnormalities. In other instances, the device is adapted for screening of driving under influence (DUI) or Driving While Intoxicated (DWI) by performing multiple eye tests resulting in field screening of DUI or DWI cases.

### FIELD OF THE INVENTION

The present invention relates a portable ocular response testing device including a portable housing, a plurality of lights for generating visible light within two eye spaces of the portable housing, a controller for operating the lights according to a prescribed pattern, and an imaging system to capture images of the eyes under varying lighting conditions to evaluate an ocular response of a user to the lighting conditions. More particularly, in some instances the present invention relates to a portable device in the field of ophthalmology for complete assessment of optic nerve defects producing Marcus-Gunn pupil, detection and quantification of color blindness, and detection and follow up of eye abnormalities. In another instance, the present invention relates generally to a device for screening of driving under influence (DUI) or Driving While Intoxicated (DWI) by performing multiple eye tests resulting in field screening of DUI or DWI cases.

### BACKGROUND

It is generally known in the medical field that various conditions affecting a patient can be detected by exposing one or both eyes to a prescribed lighting condition and measuring the resulting reflex or response of the eyes to the lighting condition. In one example, U.S. Pat. No. 7,874,675 by Konan Medical USA, Inc. discloses a device for pupillary reflex imaging in which images are taken of the pupillary reflex to a series of light flashes, each lasting approximately 0.6 seconds in duration. In another example, U.S. Pat. No. 8,951,046 by Sync-Think, Inc. discloses a desktop based opto-cognitive system arranged for cognitive assessment in which images on a single display screen are presented to both eyes of a patient and an imaging system detects gaze direction of the eyes to evaluate cognitive ability in the patient. In both instances, the devices are not well suited to be readily portable between various testing locations. Furthermore, both devices are limited in the conditions which they are able to evaluate.

Relative Afferent Pupillary Defect:

Relative Afferent Pupillary Defect (RAPD) also known as Marcus-Gunn pupil, is a quantifiable finding that may result from a variety of physiological conditions that affect optic nerve. Commonly there exists three tests that can be used for evaluating RAPD, known as (a) Swinging-flashlight test, (b) Brightness Saturation Test, and (c) Color Discrimination Test. Here we describe each test briefly:

a. Swinging-Flashlight Test: Briefly, this test is performed in a dimly lit room, using a strong light source. Pupillary reactions are observed as the light shines in one eye. Normally, when either eye is exposed to direct light, both eyes will constrict. In an individual with RAPD, shining light into an unaffected eye will cause both pupils to constrict, while shining light into the affected eye will yield a diminished constrictive response in both eyes. There are significant drawbacks associated with this test. The major drawback is the subjectivity of the examiner's opinion about the variations of pupil size and speed of response to light (which both are important parameters in evaluating the state of RAPD). Also, the unsymmetric test situation for either eye, makes the test procedure unreliable for mild RAPDs. Although the response of each eye to light stimuli should be measured independently and in isolation, the ability of swinging-flashlight test in providing this condition is limited because both patient's eyes should be open to let doctor check each pupil size change. Moreover, the ambient light in the room affects the amount of received light by each eye which makes results prone to error.

b. Brightness Saturation Test (BST): BST is used along with swinging-flashlight test to quantify RAPD. This method is to place a series of neutral density filters in front of the intact eye to change the light intensity, and to repeat the swinging-flashlight test. More particularly, this method is performed by increasing the density of the filters in front of the intact eye until the defective eye's constriction is observed to be the same level as its impaired direct reflex.

c. Color Discrimination Test (CDT): CDT is used along with swinging-flashlight test and BST to quantify RAPD. This method is to place a red object in front of one eye and to ask the patient to choose the most similar color to the object from the color bar in front of the other eye. The amount of discrepancy among the two reds, results in quantification of RAPD. Similarly, due to the subjective and verbal communication between patient and doctor, determination of exact amount of difference between perceived values of the red object by each eye is not quite possible.

Color Blindness:

A significant percentage of the human population is affected by Color Blindness. The person with color blindness is unable or is limited to see color, or distinguish color differences, under normal lighting conditions. Screening and more detailed quantitative tests are developed to detect a color vision deficiency and determine the type and severity of color blindness.

a. Color Blindness Test (CBT): Ishihara Color Vision Test is the most widely used test to detect color blindness in a patient. The patient should be in a room with the normal daylight. The examiner asks the patient to find and read a random number in a page which is covered with many dots of various colors, brightness and sizes. The complete test includes checking 38 different images each in one page. Unlike the people with normal vision, color blind patients are not able to find a number at all or they see a different number. Although it is one of the most common color blindness detection tests however, it can not be used for checking young children's vision; because they can not recognize different numbers and correct communication with them is not always possible. Farnsworth-Munsell 100 Hue Test is another popular test which makes quantification of color blindness possible. A patient should sort 400 small colored disks in a special order and the results will be compared to the standard known set. The difference between patient's results and the standard set determines the amount of color blindness. Other than the long time needed to perform this test which makes patients tired, the ambient light may affect the patient's perception of colors and make the results prone to errors. Several problems such as 1— human errors in measuring pupil size using naked eye, 2— variations in testing condition such as the changes in ambient light, 3— not controlled and non-intentional changes in penlight light intensity due the variations in batteries' charge and etc., resulted in a long sought but unfulfilled need for apparatus, methods and systems that automatically assess and quantify ophthalmologic biomarkers of Marcus-Gunn and color blindness. Moreover, after performing certain eye surgeries, patients need to be monitored regularly with short intervals. Limited access to ophthalmologist due to busy appointments imposes a challenge on getting the high standard health service that patients deserve.

Also, patients who live in remote areas have very limited access to specialists and cannot be routinely checked up. All of these, signify the need for development of an affordable and portable device which in addition to ophthalmologists, patients in remote areas can have access to and use it easily. Using this solution, patients would be able to take their eyes' images at home and share images and test results with their doctors through a secured cloud platform online. Such a solution not only benefits patients in remote areas, but also helps technicians and non-highly skilled employees in doctors' offices perform the test and save doctors' time too. Not only this solution speeds up the time that doctors need to monitor their patients and check their bio-markers remotely but also it increases the accuracy of each of mentioned tests. Digital archiving of the test results lets doctors track the progression of patients' diseases or treatments over time easily.

DUI and DWI

Traffic accidents are predominantly caused by DUI or DWI; for people in Europe between the age of 15 and 29, DUI is one of the main causes of mortality. According to the National Highway Traffic Safety Administration, DUI and alcohol related crashes cause approximately $37 billion in damages annually. DUI or DWI are not only limited to alcohol consumption, but it also includes the consumption of recreational drugs such as cannabis products such as marijuana or hashish, as well as prescription drugs such as opioids and benzodiazepines.

Drugs, including alcohol, have a profound effect upon human eye movement. There are now several studies which have demonstrated this and that is why one of the main field sobriety tests being performed by authorities in DUI or DWI cases is the evaluation of eyes which generally includes tests of equal eye size, convergence, nystagmus, and smooth pursuit. The main problem in performing the test is subjectivity of the results based on the experience of the police officer, the validity of the test results, and the potential of recording the results to be admissible in court.

There are lots of publications in the literature studying the effect of alcohol and drug consumption on different eye abnormalities. However, as the tests are commonly being performed partially and in a very subjective manner with no methods of recording the results, other than taking notes, the eye tests have not yet completely demonstrated their actual importance and accuracy in DUI or DWI cases.

There is a need for improved methods and systems for DUI or DWI field screening, which facilitates the use, while being able to simultaneously analyze and record the screening results.

### SUMMARY OF THE INVENTION

One objective of the present invention is to provide an ocular response testing device which is readily portable for performing ocular response tests in a variety of testing environments.

Another objective of the present invention is to provide an ocular response testing device which is capable of performing a variety of different ocular response tests.

According to one aspect of the present invention there is provided an ocular response testing device for testing eyes of a patient, the testing device comprising:

a portable housing arranged to be supported in proximity to the eyes of the patient, the housing including two eye spaces which are isolated from one another for independent interaction with the eyes of the patient respectively;

an infrared light generator on the housing within each eye space for illuminating the eye spaces respectively with infrared light;

an imaging system on the housing associated with both eye spaces for capturing images of both eyes of the patient when aligned with the two eye spaces for illumination by the infrared light generator respectively;

a visible light source on the housing in association with each eye space for selectively producing visible light within the eye spaces respectively; and

a controller supported on the housing which is operable to illuminate the visible light source within each eye space according to a prescribed pattern and to capture images of both eyes in response to said prescribed pattern according to a prescribed test.

The testing device may further include a portable hand-held computer device remote from the portable housing and adapted for wireless communication with the controller of the portable housing such that the testing device is operable to perform a plurality of different prescribed tests in which each prescribed test comprises a respective prescribed pattern of operating the visible light source and of capturing images using the imaging system. Preferably the computer device includes a display and operator inputs so as to be arranged to direct which prescribed test is performed by the controller of the portable housing in response to instructions input on the computer device.

Preferably the testing device also includes a portable power source supported on the portable housing for operating the infrared light generator, the imaging system, the visible light source and the controller.

When the controller is operable to perform a plurality of different prescribed tests, each comprising a respective prescribed pattern of operating the visible light source and of capturing images using the imaging system, the portable housing may further comprise an external display supported externally on the housing and operator inputs supported externally on the housing so as to be arranged to direct which prescribed test is performed by the controller of the portable housing in response to instructions input on the operator inputs and display results of a prescribed test on the external display. The external display may be provided in addition to or instead of the portable hand-held computer device remote from the portable housing.

When the testing device further comprises operator inputs supported externally on the housing for instructing operation of the controller to conduct a prescribed test, an external display supported externally on the housing for displaying results of a prescribed test thereon, and a portable power source supported on the housing for operating the infrared light generator, the imaging system, the visible light source, the controller and the external display, the portable housing is preferably sufficiently portable to be arranged to be carried on a head of a patient.

Another objective of the present invention is to eliminate the limitations in conventional testing procedures for measuring afferent eye defects and color blindness.

In one embodiment, this objective is accomplished by a device comprising two or more imaging devices which will record pupil changes of each eye accordingly. The dimmable and controllable wide-spectrum Light Emitting Diode (LED) light(s) are placed on each side of patient's eyes, such that the light rays are parallel with each eye. In a similar design the wide-spectrum LEDs) can be replaced with one or more color LED as a combination, such that it produces the same functionality. A mechanical frame holds the camera and LEDs in front of each eye. The position of these lights is determined based on ophthalmology practices which emphasize on parallelness between the emitted light beams and eye surface. Moreover, the mechanical frame is designed such that each eye is kept in full isolation from the other eye. It means that no light can be sensed by the contralateral eye when LEDs are emitting light to the right or the left eye. Also, 4 infrared LED lights are positioned in 4 sides of each camera such that the beams shine in the eye and reflect back toward the camera. To control the intensity, wavelength, and frequency of LED's light, well-known algorithm such as pulse width modulation (PWM) can be used. However, previous research has shown that there is a nonlinear relationship between temperature and Amount of consumed current by LEDs, hence the intensity of light. Therefore, using PWM algorithm or similar methods in a feedforward system does not guarantee the generation of the same amount of light intensity and/or color in different temperatures. This issue becomes more important when LEDs and related circuitries are being used for longer periods of time and/or in different environmental conditions with high temperature difference.

Moreover, due to the difference in morphological properties of each person's face, size of eyelashes and other conditions, the amount of light received by each person's' eye can be quite different even using the same instrument. To solve this problem, one or more light sensors are put in each isolated space in front of each eye. These sensors convert the amount of light intensity and wavelength to electrical signals. Electrical signals are fed back to a robust control algorithm and modify the parameters which are being used to generate PWM signals and other control signals in a driving circuit. This method results in formation of a controllable system that generates desired amount of light intensity and color by each LED accurately.

A portable apparatus, and its related methods and systems for automatic assessment and quantifying eye diseases and abnormalities including but not limited to Marcus-Gunn pupil, color blindness, hypopyon and hyphema. The proposed device is able to take and record pictures from patient's eyes to be used for patient monitoring and tracking of diseases progression. The apparatus may utilize a combination of light sources, sensors, cameras, and control algorithms for quantified measurements and assessment of eyes images to let doctor diagnose, compare, and follow up Marcus-Gunn pupil, color blindness, and other eye abnormalities in real-time.

The testing device preferably includes programming instructions stored on the testing device which is directed towards a plurality of different, prescribed tests each having a prescribed pattern of operation the visible light source, the prescribed tests including at least one RAPD test arranged to evaluate for Relative Afferent Pupillary Defect, at least one Color Blindness test arranged to evaluate color blindness and at least one eye abnormality test arranged to detect eye structure abnormalities.

The testing device may further comprises: a set of driving circuitries and control algorithms for changing the light spectrum of the visible light sources; and a timing system and programming instructions for the controller such that the controller is arranged to operate the visible light source, the infrared light generator and the imaging system to stimulate the eyes of the patient and capture images according to a Marcus Gunn Examination; the programming instructions being arranged to: (i) turn on the infrared light generator to illuminate both eye spaces; (ii) turn on the visible light Source to generate wide-spectrum, white light in a first one of the eye spaces for 2-3 seconds; (iii) capture images from both eye spaces for storage on a storage device of the controller in real time; (iv) turn on the visible light source to generate wide-spectrum, white light in a second one of the eye spaces for 2-3 seconds while turning off the visible light source in the first eye space; (v) capture images from both eye spaces for storage on a storage device of the controller in real time; (vi) process the resulting captured images so as to compare direct and indirect response of pupil and determine pupil size and shape changes; and (vii) display the processed results to the operator on a display of the testing device.

The testing device may be further adapted to perform a Brightness Saturation Test on a healthy eye relative to an abnormal eye of the patient. In this instance, the testing device further comprises: a set of driving circuitries and control algorithms for changing the light spectrum of the visible light sources; and an operator input for manually adjusting intensity of the visible light sources; a timing system and programming instructions for the controller such that the controller is arranged to operate the visible light source, the infrared light generator and the imaging system to stimulate the eyes of the patient and capture images according to a Brightness Saturation Test; the programming instructions being arranged to: (i) identify a first one of the eye spaces as being associated with the healthy eye and a second one of the eye spaces as being associated with the abnormal eye; (ii) turn on the infrared light generator to illuminate both eye spaces with infrared light; (iii) turn on the visible light source associated with the second eye space and set an intensity of the visible light produced by the visible light source according to a prescribed value; (iv) concurrently and continuously capturing images from both eye spaces in real-time; (v) determine the size of pupil of the abnormal eye in captured images associated with the second eye space in real-time; (vi) turn off the visible light sources while maintaining operation of the infrared light generator; (vii) turn on the visible light source associated with the first eye space and set an intensity of the visible light produced by the visible light source according to a prescribed value; (viii) concurrently and continuously capturing images from both eye spaces in real-time; (ix) determine pupil size of the healthy eye in captured images associated with the first eye space in real-time; (x) measure and record light intensity levels continuously in the first eye space associated with the healthy eye while dimming the visible light source in the first eye space until the determined size of the pupil in captured images from the first eye space become the same as the determined size of the pupil in captured images of the second eye space associated with the abnormal eye in the presence of the visible light at the prescribed value; (xi) determine a difference in light intensity between the prescribed value of the visible light in the second eye space and the measured light intensity level in the first eye space when the pupils in the captured images from both eye spaces have the same size; and (xii) display results of the prescribed test, including the determined difference in light intensity, to an operator of the testing device.

The testing device may be further adapted to perform a Color Discrimination Test on a first eye and a second eye of the patient. In this instance, the testing device further comprises: a set of driving circuitries and control algorithms for producing red visible light and for changing an intensity of the red visible light of the visible light sources; and an operator input for manually adjusting the intensity of the red visible light produced by the visible light sources; a timing system and programming instructions for the controller such that the controller is arranged to operate the visible light source, the infrared light generator and the imaging system to stimulate the eyes of the patient and capture images according to a Color Discrimination Test; the programming instructions being arranged to: (i) identify a first one of the eye spaces as being associated with the first eye and a second one of the eye spaces as being associated with the second eye; (ii) turn on the infrared light generator to illuminate both eye spaces with infrared light; (iii) turn on the visible light sources to produce red visible light in the first eye space; (iv) set an intensity of the red visible light according to a prescribed value; (v) concurrently and continuously capturing images from both eye spaces in real-time; (vi) determine the size of pupil of the first eye in captured images associated with the first eye space in real-time; (vii) turn off the visible light source generating the red visible light; (viii) turn on the visible light sources to produce red visible light in the second eye space; (ix) concurrently and continuously capturing images from both eye spaces in real-time; (x) determine pupil size of the second eye in captured images associated with the second eye space in real-time; (xi) measure and record red light intensity levels continuously in the second eye space associated with the second eye while dimming the red visible light produced by the visible light source in the second eye space until the determined size of the pupil in captured images from the second eye space becomes the same as the determined size of the pupil in captured images of the first eye space associated with the first eye in the presence of the red visible light at the prescribed value; (xii) determine a difference in red light intensity between the prescribed value of the red visible light in the first eye space and the measured red light intensity level in the second eye space when the pupils in the captured images from both eye spaces have the same size; and (xiii) display results of the prescribed test, including the determined difference in red light intensity, to an operator of the testing device.

The testing device may be further adapted to perform a Color Blindness Test on a first eye and a second eye of the patient, the testing device further comprising: a set of driving circuitries and control algorithms for producing red visible light and for changing an intensity of the red visible light of the visible light sources; and an operator input for manually adjusting the intensity of the red visible light produced by the visible light sources; a timing system and programming instructions for the controller such that the controller is arranged to operate the visible light source, the infrared light generator and the imaging system to stimulate the eyes of the patient and capture images according to a Color Discrimination Test; the programming instructions being arranged to: (i) turn on the infrared light generator to illuminate both eye spaces with infrared light; (ii) turn on the visible light source associated with both eye spaces of the housing; (iii) adjusting a wavelength of the visible light through a range of different wavelength values representing different colors in steps accordingly to a prescribed step size and prescribed time interval; (iv) concurrently and continuously capturing images from both eye spaces in real time; (v) determining pupil size of each eye in the captured images associated from both eye spaces; (vi) generating a report demonstrating pupil size of each eye in response to each different color represented by the different wavelength values.

The testing device may be further adapted to perform at least one eye abnormality test, the controller including programming instructions arranged to: (i) capture images from both eye spaces of eyes illuminated by one or both of the infrared light generator and the visible light source; and (ii) compare captured images from both eye spaces to respective ones of previously captured images from the eye spaces to identify differences in eye structures in each eye relative to the same eye in the previously captured images related to abnormalities in the eyes.

When the imaging system comprises a camera in alignment with each eye space for capturing images of a respective eye of the patient, and the infrared light generator preferably comprises a plurality of infrared light sources at circumferentially spaced apart locates about each camera.

In some embodiments, the testing device further includes at least one manually operable input mounted externally on the portable housing which is arranged to vary intensity of the visible light produced by the visible light source.

In some embodiments, the testing device further includes at least one manually operable input mounted externally on the portable housing which is arranged to vary wavelength of the visible light produced by the visible light source.

In some embodiments, the visible light source within each eye space is preferably arranged to produce uniform visible light with no fixation point within each eye space.

Preferably the visible light source associated with each eye space comprises a wide-spectrum light emitting diode which is controllable so as to be dimmable and adjustable in wavelength and which is positioned within the respective eye space so as to generate light rays parallel to a respective eye received in the eye space.

According to another important independent aspect of the present invention there is provided a testing device further comprising:

a driving circuit associated with the visible light source within each eye space of the portable housing which receives control signals from the controller; and

a light sensor supported on the portable housing within each eye space so as to be arranged to convert visible light produced by the visible light source within each eye space into electrical signals associated with the respective eye spaces which are representative of light intensity and light wavelength;

the controller being arranged to modify parameters used to generate controls signals associated with each driving circuit responsive to the electrical signals from the light sensors such that a characteristic of the visible light produced by the visible light source within each eye space is maintained at levels specified by the prescribed test.

The controller is preferably arranged to modify parameters used to generate control signals associated with intensity and/or wavelength of the visible light produced by the visible light sources.

When the imaging system comprises a camera in alignment with each eye space for capturing images of a respective eye of the patient, the light sensors preferably comprise a plurality of light sensors within each space which are located at diametrically opposed locations about the respective camera.

In one general aspect, a pupillary response scanning device and methods for use thereof in performing swinging flashlight test, brightness saturation test, color discrimination test, and color blindness test is described. The pupillary response scanning device may include: a housing configured to provide a first isolated eye enclosure for a first eye and a second isolated eye enclosure for a second eye of a patient; one or more visible light sources, one or more infrared light sources, one or more imaging devices in each isolated eye enclosure; and a control system that may be configured for manipulating the visible light sources, infrared light sources and the imaging devices. The housing may be configured to keep the eyes isolated from environment and one another to prevent light emitted from sources other than those inside an eye enclosure from entering the eye enclosed in that eye enclosure.

In another general aspect, a method is described for ocular assessment that may include steps of: providing a pupillary response scanning device that may include: a housing configured to provide a first isolated eye enclosure for a first eye and a second isolated eye enclosure for a second eye of a patient; one or more visible light sources, one or more infrared light sources, one or more imaging devices in each isolated eye enclosure; and a control system that may be configured for manipulating the visible light sources, infrared light sources and the imaging devices; continuously illuminating the first and second eyes of the patient with infrared light from the one or more infrared light sources during testing; capturing at least one reference image of the first and the second eye with the one or more imaging devices; illuminating the first eye of the patient with visible light from the one or more visible light sources in the first eye enclosure for a predetermined duration; concurrently capturing at least one first test image of the first and the second eyes with the one or more imaging devices; subjecting the first and the second eyes to a dark adaptation period under infrared illumination from the one or more infrared light sources; illuminating the second eye of the patient with visible light from one or more visible light sources in the second eye enclosure for a predetermined duration; concurrently capturing at least one second test image of the first and the second eyes with the one or more imaging devices; and transmitting the reference images, the first and the second test images to the control system, wherein the control system is configured to process the transmitted images to identify an affected eye and a healthy eye.

According to some implementations, the method for ocular assessment may further include the steps of: illuminating the affected eye with visible white light from one or more visible light sources for a predetermined duration; concurrently capturing at least one first test image of the affected eye with the one or more imaging devices; subjecting the affected eye and the healthy eye to a dark adaptation period under infrared illumination from the one or more infrared light sources; illuminating the healthy eye with visible white light from the one or more visible light sources for a predetermined duration with a predetermined initial intensity; gradually changing the intensity of the visible white light illuminated into the healthy eye; concurrently capturing consecutive test images of the affected eye for each light intensity with the one or more imaging devices; transmitting the first test image and the consecutive test images to the control system.

According to one implementation, the control system may be configured to: determine a pupil size for the affected eye in direct white light application from the first test image; determine a pupil size for the affected eye in consensual white light application for each light intensity from the consecutive test images; and find a light intensity at which the pupil size of the affected eye in direct white light application is equal to pupil size for the affected eye in consensual white light application.

According to other implementations, the method for ocular assessment may further include the steps of: illuminating the affected eye with visible red light from one or more visible light sources for a predetermined duration; concurrently capturing at least one first test image of the affected eye with the one or more imaging devices; subjecting the affected eye and the healthy eye to a dark adaptation period under infrared illumination from the one or more infrared light sources; illuminating the healthy eye with visible red light from the one or more visible light sources for a predetermined duration with a predetermined initial intensity; gradually changing the intensity of the visible red light illuminated into the healthy eye; concurrently capturing consecutive test images of the affected eye for each light intensity with the one or more imaging devices; transmitting the first test image and the consecutive test images to the control system.

According to one implementation, the control system may be configured to: determine a pupil size for the affected eye in direct red light application from the first test image; determine a pupil size for the affected eye in consensual red light application for each light intensity from the consecutive test images; and find a light intensity at which the pupil size of the affected eye in direct red light application is equal to pupil size for the affected eye in consensual red light application.

In another general aspect, a method for color blindness test is described that may include the steps of: providing a pupillary response scanning device that may include: a housing configured to provide a first isolated eye enclosure for a first eye and a second isolated eye enclosure for a second eye of a patient; one or more visible light sources, one or more infrared light sources, one or more imaging devices in each isolated eye enclosure; and a control system that may be configured for manipulating the visible light sources, infrared light sources and the imaging devices; continuously illuminating the first and second eyes of the patient with infrared light from the one or more infrared light sources during testing; capturing at least one reference image of the first and the second eye with the one or more imaging devices; illuminating the healthy eye with visible light from the one or more visible light sources for a predetermined duration with a predetermined initial wavelength; gradually changing the wavelength of the visible light from an initial wavelength to a final wavelength; concurrently capturing consecutive test images of the first and the second eyes for each wavelength with the one or more imaging devices; and transmitting the first test image and the consecutive test images to the control system. The control system may be configured to determine a pupillary reactivity for the first eye or the second eye to direct light application for each wavelength.

In some applications, the pupillary response scanning device may further include a first adjustment knob and a second adjustment knob. The first adjustment knob may be configured for changing the intensity and/or the wavelength of the one or more visible light sources in the first isolated eye enclosure. The second adjustment knob may be configured for changing the intensity and/or the wavelength of the one or more visible light sources in the second isolated eye enclosure.

Another objective of the present invention is to eliminate the limitations in conventional testing procedures for measuring DUI and DWI screening.

In one embodiment, this objective is accomplished by a DUI or DWI screening system using eye test which is provided with a portable wireless device and associated methods and software to enable the user to perform eight different eye tests, individually. The system includes imaging system as well as eye stimulating system to stimulate each eye in a pre-defined pattern and record the reaction of the pupil. The user commands to run the tests is being sent to the device wirelessly, and the recorded images and videos will be stored in the hand-held device, which includes the software. The software stores and shows the test result to the user.

In this instance, the testing device may be adapted to preform a plurality of screening tests selected from the list consisting of: (i) a resting Nystagmus eye test; (ii) a Horizontal Gaze Nystagmus eye test; (iii) a Vertical Gaze Nystagmus eye test; (iv) a Lack of Smooth Pursuit eye test; (v) an Equal Pupil eye test; (vi) a Nystagmus at Maximum Deviation eye test; (vii) a Nystagmus Prior to 45 Degrees eye test; and (viii) a Non-convergence eye test.

When the screening test includes a resting Nystagmus eye test, the testing device preferably further comprises programming instructions for the controller adapted to: (i) turn on the infrared light generator in each eye space while maintaining the eye spaces in darkness; (ii) capture images to generate a stream of eye images associated with each eye space; (iii) concurrently in real-time, identify a pupil in the captured images of each stream of eye images and track a corresponding movement of the pupil for a prescribed duration; (iv) determine if a resting Nystagmus condition is present in the streams of eye images; (v) generate a notification through an output of the testing device if the resting Nystagmus condition is determined to be present.

When the screening test includes an Equal Pupil eye test, the testing device preferably further comprises programming instructions for the controller adapted to: (i) turn on the infrared light generator in each eye space while maintaining the eye spaces in darkness; (ii) capture images of respective eyes of the patient associated with each eye space; (iii) identify a pupil in the captured images of each eye space and determine a size of the identified pupil within each eye space; (iv) compare a difference between the size of the identified pupils of the two eye spaces to an equal pupil criteria to determine if a non-equal pupil condition is present in the eye images; and (v) generate a notification through an output of the testing device if the non-equal pupil condition is determined to be present.

The visible light source of the testing device may comprise a sequence of light emitting diodes supported within each eye space in spaced apart relation with one another.

The sequence of light emitting diodes within each eye space may comprise a horizontal row of light emitting diodes. In this instance, the screening test may include a Horizontal Gaze Nystagmus test in which the testing device further comprises programming instructions for the controller adapted to: (i) turn on the infrared light generator in each eye space; (ii) illuminate the light emitting diodes in sequence with one another along the horizontal row of light emitting diodes; (iii) capture images to generate a stream of eye images associated with each eye space while the light emitting diodes are sequentially illuminated; (iv) identify a pupil in the captured images of each stream of eye images and track a corresponding movement of the pupil; (v) compare the movement from each stream of eye images to a horizontal gaze criteria to determine if a Horizontal Gaze Nystagmus condition is present in the eye images; and (vi) generate a notification through an output of the testing device if the Horizontal Gaze Nystagmus condition is determined to be present.

The sequence of light emitting diodes within each eye space may also comprise a vertical row of light emitting diodes. In this instance, the screening test may include a Vertical Gaze Nystagmus test in which the testing device further comprises programming instructions for the controller adapted to: (i) turn on the infrared light generator in each eye space; (ii) illuminate the light emitting diodes in sequence with one another along the horizontal row of light emitting diodes; (iii) capture images to generate a stream of eye images associated with each eye space while the light emitting diodes are sequentially illuminated; (iv) identify a pupil in the captured images of each stream of eye images and track a corresponding movement of the pupil; (v) compare the movement from each stream of eye images to a vertical gaze criteria to determine if a Vertical Gaze Nystagmus condition is present in the eye images; and (vi) generate a notification through an output of the testing device if the Vertical Gaze Nystagmus condition is determined to be present.

The sequence of light emitting diodes within each eye space may also follow a generally annular path in which some of the light emitting diodes are horizontally spaced apart from one another and some of the light emitting diodes are vertically spaced apart from one another. In this instance, the screening test may include a Lack of Smooth Pursuit test in which the testing device further comprises programming instructions for the controller adapted to: (i) turn on the infrared light generator in each eye space; (ii) illuminate the light emitting diodes in sequence with one another along the generally annular path of light emitting diodes; (iii) capture images to generate a stream of eye images associated with each eye space while the light emitting diodes are sequentially illuminated; (iv) identify a pupil in the captured images of each stream of eye images and track a corresponding movement of the pupil; (v) compare the movement from each stream of eye images to a smooth pursuit criteria to determine if a Lack of Smooth Pursuit condition is present in the eye images; and (vi) generate a notification through an output of the testing device if the Lack of Smooth Pursuit condition is determined to be present.

When the screening test also includes a Nystagmus at maximum deviation test, the testing device preferably further comprises programming instructions for the controller adapted for each eye space to: (i) turn on the infrared light generator in the eye space; (ii) activate the visible light source to illuminate the eye space for a first duration; (iii) inactivate the visible light source such that the eye space is in darkness for a second duration; (iv) activate the visible light source to illuminate the eye space for a third duration; (v) capture images to generate a stream of eye images associated with the eye space during the first duration, the second duration and the third duration; (vi) identify a pupil in the captured images of the stream of eye images for the eye space and track a corresponding movement of the pupil; (vii) compare the movement from the stream of eye images to a maximum deviation criteria to determine if a Nystagmus at maximum deviation condition is present in the eye images; and (viii) generate a notification through an output of the testing device if the Nystagmus at maximum deviation condition is determined to be present.

In some embodiments when the imaging system of the testing device comprises a camera in each eye space for alignment with the respective eye of the patient, the visible light source may comprise a left light emitting diode and a right light emitting diode in each eye space at respective diametrically opposing sides of the camera. In this instance, the screening test may include a Nystagmus prior to 45 Degrees test in which the testing device further comprises programming instructions for the controller adapted for each eye space to: (i) turn on the infrared light generator in the eye space; (ii) activate the left light emitting diode for a first duration while the right light emitting diode is inactive; (iii) inactive both left and right light emitting diodes for a second duration; (iv) activate the right light emitting diode for a third duration while the left light emitting diode is inactive; (v) capture images to generate a stream of eye images associated with the eye space during the first duration, the second duration and the third duration; (vi) identify a pupil in the captured images of the stream of eye images for the eye space and track a corresponding movement of the pupil; (vii) compare the movement from the stream of eye images to a 45 degree criteria to determine if a Nystagmus prior to 45 degrees condition is present in the eye images; and (viii) generate a notification through an output of the testing device if the Nystagmus prior to 45 degrees condition is determined to be present.

In some embodiments when the imaging system of the testing device comprises a camera in each eye space for alignment with the respective eye of the patient, the visible light source may comprise a convergence light emitting diode in each eye space at a location which is laterally inwardly relative to the respective camera such that the two convergence light emitting diodes appear to converge with one another from a perspective of the eyes of the patient. In this instance, the screening test may include a Non-convergence test in which the testing device further comprises programming instructions for the controller adapted for each eye space to: (i) turn on the infrared light generator in the eye space; (ii) capture images to generate a stream of eye images associated with each eye space; (iii) simultaneously activate both convergence light emitting diodes for a prescribed duration; (iv) identify a pupil in the captured images of the stream of eye images for both eye spaces and track a corresponding movement of the pupils; (v) compare the movement from the stream of eye images to a non-convergence criteria to determine if a Non-convergence condition is present in the eye images; and (vi) generate a notification through an output of the testing device if the Non-convergence condition is determined to be present.

Each light emitting diode may include a covering frame portion covering the light emitting diode including a pin hole therein such that any light emitted by the light emitting diode is pin-shaped.

### BRIEF DESCRIPTION OF THE DRAWINGS

Various embodiments of the invention will now be described in conjunction with the accompanying drawings in which:

FIG. 1 is a schematic functional block diagram of one implementation of the testing device, according to one or more aspects of the present disclosure for use as a pupillary response scanning device.

FIG. 2A illustrates a perspective view of the pupillary response scanning device.

FIG. 2B illustrates a top view of the pupillary response scanning device.

FIG. 2C illustrates a front view of the pupillary response scanning device.

FIG. 3A illustrates a logical flow of operations in one example process for performing a swinging flashlight test using the pupillary response scanning device, according to one or more aspects of the present application.

FIG. 3B illustrates a logical flow of operations in one example process for performing a brightness saturation test (BST) using the pupillary response scanning device, according to one or more aspects of the present application.

FIG. 3C illustrates a logical flow of operations in one example process for performing a color discrimination test (CDT) using the pupillary response scanning device, according to one or more aspects of the present application.

FIG. 3D illustrates a logical flow of operations in one example process for performing a color blindness test using the pupillary response scanning device, according to one or more aspects of the present application.

FIG. 4 schematically illustrates the DUI or DWI screening device according to a second embodiment of the testing device;

FIG. 5 shows the handheld device and a screenshot of the software running on it according to the second embodiment of the testing device;

FIG. 6 shows the top view of the DUI or DWI screening device according to the second embodiment of the testing device;

FIG. 7 shows the front view of the DUI or DWI screening device according to the second embodiment of the testing device;

FIG. 8 shows the schematic of the LED-driver printed circuit board according to the second embodiment of the testing device;

FIG. 9 demonstrates two of the different patterns of white LEDs on the LED-driver printed circuit board, and defined the naming convention for each white LED according to the second embodiment of the testing device;

FIG. 10 is a flowchart showing steps of Resting Nystagmus test according to the second embodiment of the testing device;

FIG. 11 is a flowchart showing steps of Horizontal Gaze Nystagmus test according to the second embodiment of the testing device;

FIG. 12 is a flowchart showing steps of Vertical Gaze Nystagmus test according to the second embodiment of the testing device;

FIG. 13 is a flowchart showing steps of Equal Pupils test according to the second embodiment of the testing device;

FIG. 14 is a flowchart showing steps of Lack of Smooth Pursuit test according to the second embodiment of the testing device;

FIG. 15 is a flowchart showing steps of Nystagmus at Maximum Deviation test according to the second embodiment of the testing device;

FIG. 16 is a flowchart showing steps of Nystagmus Prior to 45 Degrees test according to the second embodiment of the testing device;

FIG.17 is a flowchart showing steps of Non-convergence test according to the second embodiment of the testing device; and

FIG. 18 is a front view of a third embodiment of the testing device.

In the drawings like characters of reference indicate corresponding parts in the different figures.

### DETAILED DESCRIPTION

Referring to the accompany figures, there is illustrated an ocular response testing device generally indicated by reference numeral 100. The testing device 100 in each instance is arranged for testing eyes of a patient by illuminating the eyes under various lighting conditions, capturing images of the eyes, and analyzing the images to determine various conditions of the patient.

Although various embodiments of the testing device 100 are shown in the accompanying figures, the common features of the various embodiments will first be described. In each instance the device 100 includes a readily portable housing 102 which is sufficiently small and lightweight that the housing can be readily carried between different testing locations. In some instances, the housing includes straps 104 which enable the housing to be carried on the head of a person being tested. A handle 106 may also be provided such that the housing can be readily supported in a single hand of an operator in alignment with a person being tested.

The housing generally includes a front side 108 for positioning against the face of the person being tested and an opposing rear side 110 which faces away from the person being tested. A recessed cavity 112 is provided within the housing which is open to the front side. A separator 114 is provided within the cavity to separate the cavity into two hollow eye spaces 116 for alignment with respective eyes of the person being tested. Each cavity is surrounded by perimeter walls terminating at a peripheral edge at the front side of the housing which is shaped to conform to the face of the person being tested. Accordingly, when the front side of the housing is positioned against the face of the person, the two eye spaces 116 are isolated from one another and from ambient light by the peripheral walls and the separator 114 which mate with the face of the user.

An imaging system is provided in the form of two cameras 118 mounted within the two eye spaces 116 respectively. Each camera is mounted within the respective eyes space 116 at a location spaced forwardly from the front side of the housing so as to be directed forwardly towards a respective eye of the person being tested. The two cameras are approximately centred relative to the two eyes respectively and are arranged in parallel to the lines of sight of the person. The cameras 118 capture a sequence of eye images of the respective eyes of the person to produce a video stream. Optional lenses 120 are associated with each camera. The cameras are arranged to capture both infrared and visible light for recording and capturing eye images of the respective eyes of the person when illuminated with visible light or when in darkness and illuminated only by infrared light.

In a preferred embodiment, the cameras are adjustable in horizontal distance relative to one another by a positioning mechanism having a manual input supported externally of the housing. More particularly, the manual input comprises a knob supported on a top side of the portable housing which is connected by a shaft to an internal gear. The cameras are supported on laterally sliding frame portions which are laterally slidable relative to the housing. A rack of gear teeth on each sliding frame portion meshes with the internal gear such that rotating the knob in one direction causes both cameras to be slidably displaced laterally outwardly relative to the housing, away from one another, whereas rotating the knob in the other direction causes both cameras to be slidably displaced laterally inwardly towards one another. In this manner, the lateral distance between the cameras can be adjusted to correspond to the lateral distance between the eyes of the person to be tested.

An infrared light generator is provided in the form of a plurality of infrared light emitting diodes 122 mounted within each of the eye spaces. Within each eye space, the associated infrared light emitting diodes 122 are arranged at substantially evenly spaced apart positions in a circumferential direction about the respective camera 118 at a location spaced rearwardly of the front side of the housing so as to direct infrared light forwardly onto the eyes of the person when activated.

A visible light source is also provided within each eye space of the portable housing including at least one main light emitting diode for producing visible white light. Within each eye space, the visible light source may comprise a single, full/wide spectrum light emitting diode 124 at a location rearwardly of the front side of the housing within the respective eye space for directing visible light into the eye spaces or onto the eyes of the person. Alternatively, the visible light emitting diode within each eye space may comprise a plurality of different colour LEDs 126 which collectively provide a full/wide spectrum of white light.

A circuit board 128 is provided internally within the portable housing which functions as a controller and which includes a memory and a processor for executing programming instructions stored on the memory. The circuit board 128 also includes appropriate circuitry for driving all of the light emitting diodes according to a prescribed intensity, wavelength, and duration according to different testing programs being executed by the device 100. The circuit board includes individual driving circuits for each light emitting diode or array of light emitting diodes. The circuit board also provides the function of a controller on the portable housing including a memory 50 and a processor 52 for executing programming instructions stored on the memory.

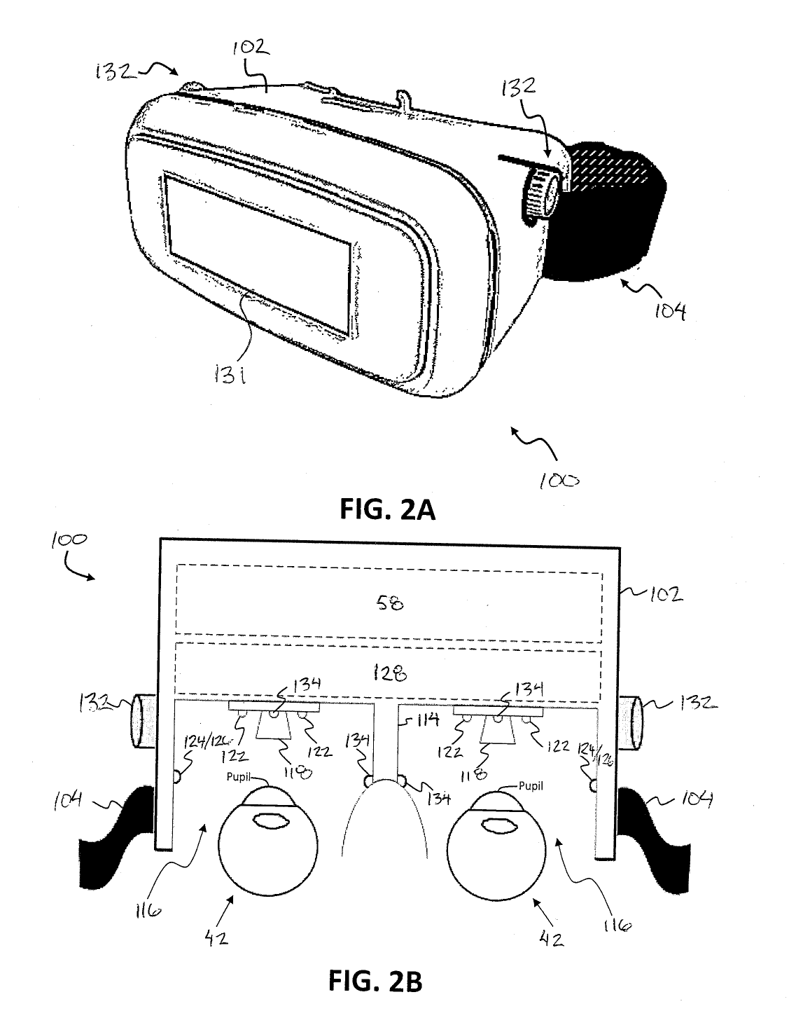

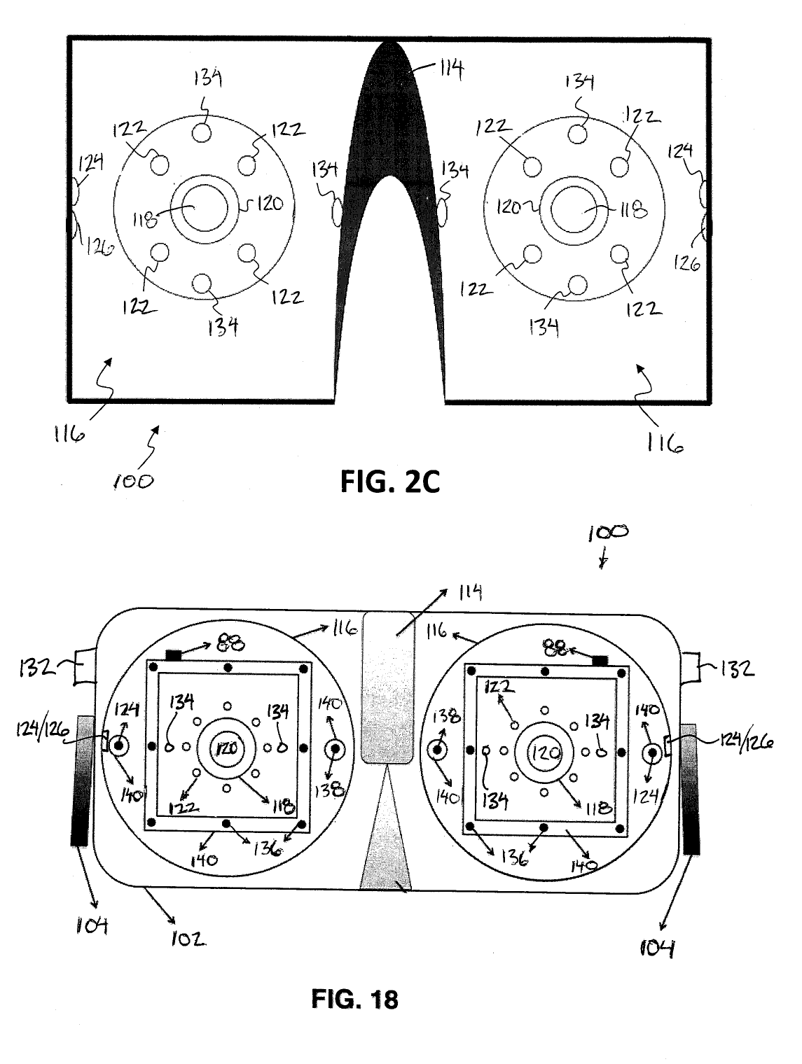

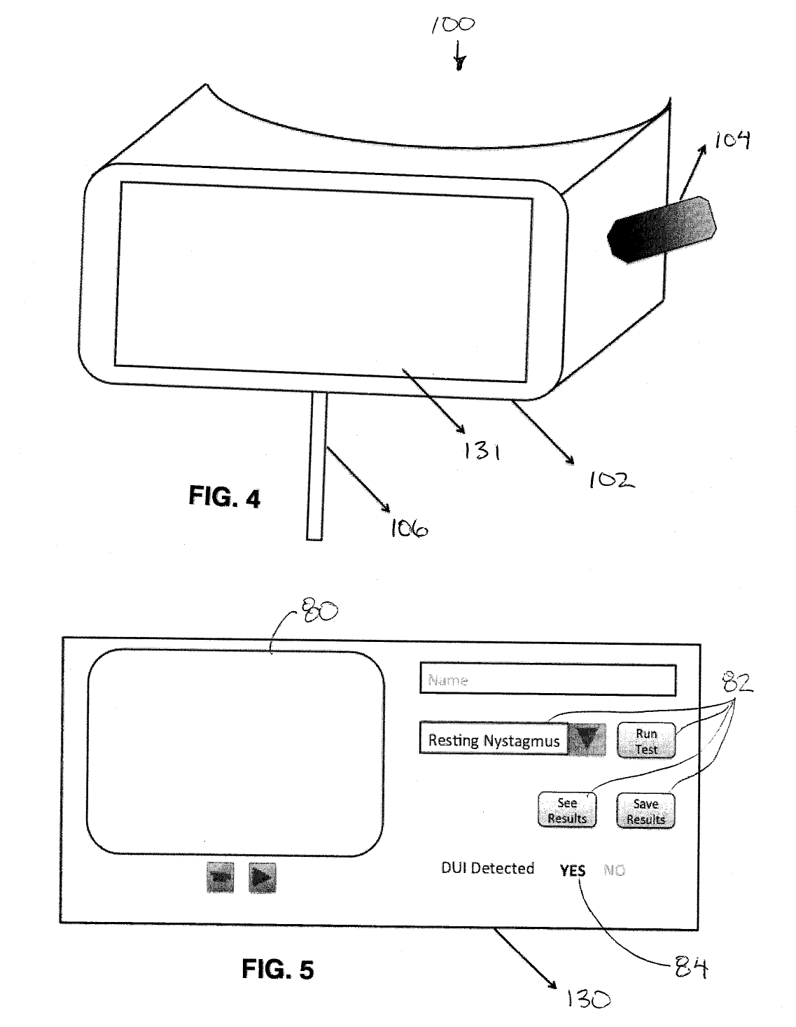

The testing device 100 may also be used together with a computer device 130 having suitable operator inputs and operator outputs for receiving instructions from an operator and displaying results to the operator. The output can be displayed on a display screen of the computer device which may be integral with the portable housing as shown by the display screen 131 in FIG. 2A or FIG. 4 or may be provided as a remotely located portable handheld device as shown in FIG. 5. In either instance the computer device 130 also includes a memory 50 and a processor 52 for executing program instructions stored on the memory. The programming instructions for executing various eye tests according to the various embodiments can be stored on the memory and executed by the processor of either one of the controller within the portable housing or the computer device 130 which may be integral or remote from the portable housing. In either instance, instructions are received from an operator through suitable inputs which cause the controller to drive activation and inactivation of selected ones of the light emitting diodes at prescribed intensities and wavelengths according to a prescribed pattern such that an image analysis algorithm which forms part of the programming instructions can analyse the captured eye images to determine if certain criteria are met which are representative of a condition of the person being tested. The captured video images together with the output analysis are typically displayed in real-time on the display of the computer device 130, and/or stored or transferred to an auxiliary computer device for subsequent analysis.

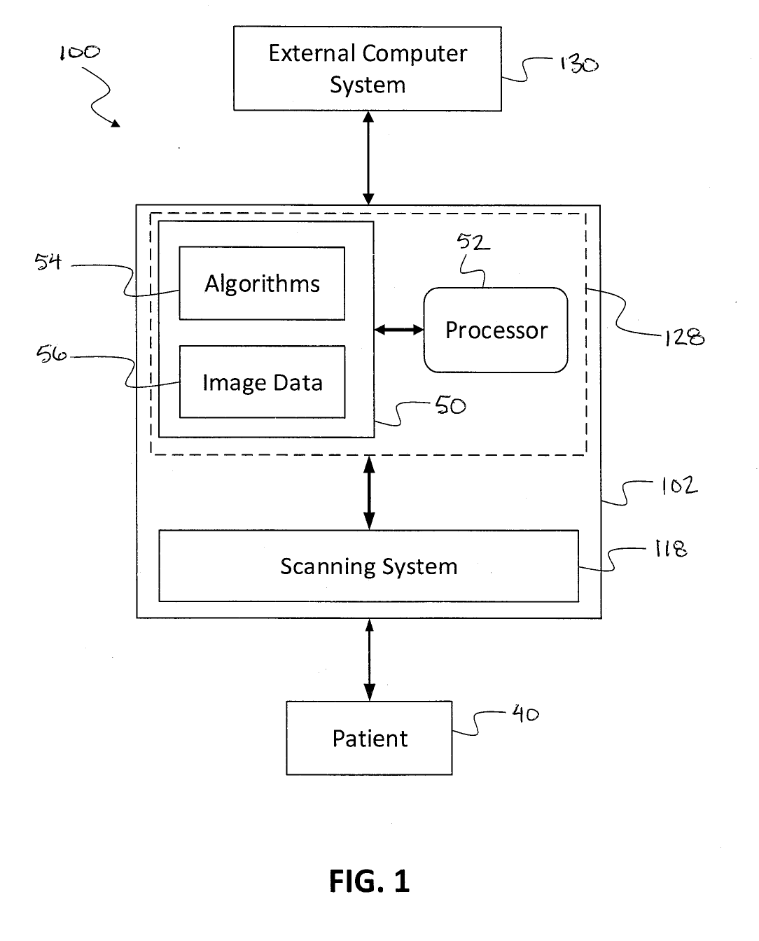

Turning now more particularly to the first embodiment of FIGS. 1 through 3, in this instance the device 100 further includes a plurality of manual operator inputs 132 in the form of control knobs supported externally on the portable housing on the rear or on laterally opposing sides of the housing for ready access by an operator or the person being tested. Each operator input 132 is associated with a respective one of the two eye spaces. The external inputs can be used as variable inputs to the controller for varying the wavelength and/or intensity of the visible light emitted by the light emitting diodes within each eye space respectively according to different eye tests being conducted.

The visible light source 124, 126 within each eye space of the portable housing is located near the front side of the housing within both eye spaces respectively. Within each eye space, the light source 124, 126 is located laterally outwardly relative to the respective camera 118 for directing visible light laterally inwardly across the eyes of the person so as to be parallel to a surface of the eyes and perpendicular to a line of sight of the eyes of the person.

In the first embodiment, the device 100 is also provided with a plurality of light sensors 134 mounted within the two eye spaces respectively. Within each eye space to light sensors are provided at diametrically opposed locations relative to the respective camera therebetween such that two light sensors and the camera are aligned in a common vertical row. An additional light sensor 134 is also provided in each eye space at a location spaced laterally inwardly towards the other eye space relative to the respective camera such that one of the light sensors is located diametrically across from the respective main visible light source 124, 126 relative to the camera within each eye space. The light sensors 134 are capable of detecting the intensity and the wavelength of the light generated within the respective eye space to convert the light into electrical signals which are fed back to the controller. The electrical signals are used to modify the control signals of the respective driving circuits of the light emitting diodes. More particularly the light sensors are used to validate whether or not the intensity and wavelength of the light emitted by the diodes corresponds to a prescribed setpoint magnitude for intensity and wavelength dictated by the programming instructions. If the actual character of the light being generated in the eye space deviates from the prescribed setpoint values, the driver signals are modified by the controller until the actual light generated by the LEDs corresponds to the prescribed setpoint values dictated by the programming instructions such that the visible light within each eye space always corresponds to and is maintained at the prescribed setpoint values dictated by the programming instructions.

In the first embodiment of FIGS. 1 through 3, the controller typically also includes a wireless communications antenna incorporated therein such that the computer device 130 in this instance is a remote, portable, handheld computer device which the operator uses to instruct the controller to perform eye tests and to view results of the tests thereon.

Turning now to the operation of the first embodiment of the testing device 100 according to FIGS. 1 through 3, the proposed device in this instance will be used to perform the tests related to 1— Relative Afferent Pupillary Defect, 2— Color blindness, 3— Eye abnormality detection.

Modes Associated with Relative Afferent Pupillary Defect (RAPD):

1— In the first mode, a smart algorithm set the parameters of the LEDs' driver circuitries such that each LED generates white light. At this point, these LEDs are called White LEDs. One of the white LEDs is turned on by the control algorithm, while the other white LED which is placed in the contralateral space is turned off. The infrared LEDs are all turned on. The reaction of both pupils will be recorded by taking a series of images using two cameras working in synchronization. After 3 seconds (or any other value which can be set by user) the first LED will be turned off, while the other one will be turned on after 5 seconds (such that they eyes have enough time to relax in the dark environment). Similarly, the reaction of both pupils will be recorded using two cameras simultaneously. The intensity and frequency of light at each moment is being monitored by light sensors and the control system. The recordings will be transferred to the monitoring device wirelessly. The drawing of the pupil size change with respect to time as well as the changes velocity, demonstrate the presence of RAPD. The quantification of RAPD is determined based on the integration of the results of the this and next stages.

2— In the second mode, after targeting the eye with RAPD, instead of using common neutral density filters and performing the BST test manually, a smart algorithm automates the test. A smart algorithm sets the parameters of the LEDs' driver circuitries such that each LED generates white light at a certain user-specified intensity. At this point, these LEDs are called White LEDs. First, the size (or any other biomarker) of pupil will be measured and recorded in the presence of a predetermined value (set by doctor or specialist) of light intensity generated by white LEDs. Then all lights, except the IR LEDs, will be turned off. The smart algorithm will start increasing the intensity of light, generated by white LEDs, in the contralateral side (which is the abnormal eye) and cameras take pictures continuously until the size (or any other biomarker) of the abnormal eye become the same as the recorded size (or any other biomarker) of the healthy eye. The intensity and frequency of light at each moment is being monitored by light sensors and the control system. The discrepancy between two White LED dimming levels, which is reported in the software demonstrates the result of BST test.

3— The third mode lets patients or doctors change the light intensity manually in a controllable way. A smart algorithm sets the parameters of the LEDs' driver circuitries such that each LED generates White light. At this point, these LEDs are called White LEDs. Each time, only one eye is illuminated with a predefined light intensity value, generated by White LEDs. After specific amount of time, all lights, except the IR LEDs, will be turned off. Then the doctor asks his/her patient to use the knob on the contralateral side to adjust the dimming level of White LEDs. The patient changes the light intensity until his/her perceived value of light intensity for both eyes become the same. Changing the dimming level of White LEDs also can be done by the operator (doctor, specialist, etc.) using a software installed on a hand-held (Tablet, cellphone, Laptop, etc.) device. In this case, the operator asks patient to let him/her know when two lights look the same. The intensity and frequency of light at each moment is being monitored by light sensors and the control system. This test gives doctors complementary information and quantify patient's eye abnormalities in perception of light values in both eyes. In fact, this mode complements the information and tests using mode 2.

4— In the fourth mode, instead of using manual qualitative methods and relying on verbal communications with patient for performing CDT test, a smart algorithm automates the test. A smart algorithm sets the parameters of the LEDs' driver circuitries such that each LED generates Red light. At this point, these LEDs are called Red LEDs. First, the size (or any other biomarker) of pupil will be measured and recorded in the presence of a predetermined value (set by doctor or specialist) of Red light intensity, generated by Red LEDs. Then all lights, except the IR LEDs, will be turned off. The smart algorithm will start increasing the intensity of Red light in the contralateral side and cameras take pictures continuously until the size (or any other biomarker) of the contralateral eye become the same as the recorded size (or any other biomarker) of the first eye. The intensity of Red light at each moment is being monitored by light sensors and the control system. The discrepancy between two Red LED dimming levels, which is reported in the software demonstrates the result of CDT test.

5— The fifth mode lets patients or doctors change the light intensity manually in a controllable way. A smart algorithm sets the parameters of the LEDs' driver circuitries such that each LED generates Red light. At this point, these LEDs are called Red LEDs. Each time, only one eye is illuminated with a predefined light intensity value, generated by Red LEDs. After specific amount of time, all lights, except the IR LEDs, will be turned off. Then the doctor asks his/her patient to use the knob on the contralateral side to adjust the dimming level of Red LEDs. The patient changes the light intensity until his/her perceived value of light intensity for both eyes become the same. Changing the dimming level of Red LEDs also can be done by the operator (doctor, specialist, etc.) using a software installed on a hand-held (Tablet, cellphone, Laptop, etc.) device. In this case, the operator asks patient to let him/her know when two lights look the same. The intensity of Red light at each moment is being monitored by light sensors and the control system. This test gives doctors complementary information and quantify patient's eye abnormalities in perception of light values in both eyes. In fact, this mode complements the information and tests using mode four.

Modes Associated with Color Blindness Test:

1— Automatic mode: In this mode, unlike the tests related to RAPD, both eyes receive the same light stimuli simultaneously. The patient is asked to put the device on and look forward. Both eyes will be illuminated by IR LEDS and cameras start taking pictures from both eyes concurrently. A smart algorithm start driving wide-spectrum LEDs such that the light wavelength changes from the lowest possible set values to the highest set values with a predefined step size automatically. Doing that, color of LED lights in both enclosed spaces changes over a desired range of values simultaneously. At each step, both eyes will be illuminated by a specific light color for a fixed amount of time which can be set by the operator. Light sensors measure light intensity and color at each moment in each enclosed space. The measurements will be fed-back to the control algorithm and associated circuitries for accurate generation of desired lights for both eyes. An algorithm, measures the pupil size (Diameter, area, or other measures of interest) for both eyes in the presence of each color. The change in pupil size when both eyes get exposed to different light colors (but with the same intensity), determines the variation in sensitivity of patient's eyes to different light colors. The results can be used as an alternative method for the manual and lengthy color blindness test.

2— Manual mode: In this mode, unlike the automatic mode, the operator changes the light color of LEDs him/herself when needed. The patient is asked to put the device on and look forward. Both eyes will be illuminated by IR LEDS and cameras start taking pictures from both eyes concurrently. The operator uses the knobs on the monitoring device to send command signals to a smart algorithm. Based on the operator's command, the smart algorithm generates proper control signals to be used by LEDs' driver circuitries such that each LED generates the desired light color with the right intensity. The same light will illuminate both eyes simultaneously. The time of light illumination can be set by the operator. Light sensors measure light intensity and color at each moment in each enclosed space. The measurements will be fed-back to the control algorithm and associated circuitries for accurate generation of desired lights for both eyes. An algorithm, measures the pupil size (Diameter, area, or other measures of interest) for both eyes in the presence of each color. The change in pupil size when both eyes get exposed to different light colors (but with the same intensity), determines the variation in sensitivity of patient's eyes to different light colors. The results can be used as an alternative method for the manual and lengthy color blindness test.

Modes Associated with Eye Abnormality Detection:

1— This mode takes and records images from the whole surface of the eye including pupil, iris, and sclera. An image processing algorithm is employed to use the captured images to detect, analyze and track abnormalities in short and long term. The abnormalities can include, but not limited to, nevus in the iris, inflammation in the iris (hypopyon), blood in the iris (hyphema), surgery marks, and the size, number, and pattern of blood vessels on sclera (directly related to the progression of diabetes). For example, the size and pattern of a nevus (or other measurements that the doctor is interested to perform manually using tools implemented in software) can be kept tracked and abnormalities be highlighted.

Similarly, the image processing algorithm identifies blood vessels on the surface of the eye and measures their length, diameter and pattern. A comparison algorithm compares the captured images from the same patient and the results of analysis in different instances of time to let doctors and specialists follow up the progression of each disease and the effect of drug/surgery on patient's eyes. The above and other features, processes, and advantages of the present invention will be apparent from the following drawings in conjunction with the accompanying detailed description.

FIG. 2B shows the top view of the device. There are two enclosed spaces for each eye. The same components such as Wide-spectrum LEDS, IR LEDs, Cameras, and Light sensors are places in each enclosed space. The enclosed spaces and the separator prevent entrance of external light or emitted light from LEDs to the contralateral side. The power of device can be provided by an external source or by having batteries installed on the device. The operation of the system is controlled by proper electronic and control circuitries.

FIG. 2C shows the inner-front view of the device. There is at least one camera in each space with an optional lens. IR LEDs illuminate each enclosed space. Light sensors measure the wavelength and intensity of the light inside each enclosed space. Wide spectrum LEDs, can be replaced with combinations of one or more color LEDs. Each external knob can be used by patient to change the light intensity in each enclosed space independently. Optional straps are used to hold the device in front of patient's eyes.

According to the first embodiment the ocular response testing device is a portable pupillary response scanning and recording apparatus comprising: a. An instrument consisting of a frame that makes a closed isolated space around each eye; one or more imaging device for photography of eyes including pupils and anterior segments in each space; b. One or more infrared LED lights to illuminate each eye during testing; c. One or more LEDs that can stream wide-range of visible light; d. One or more optional color LEDs to replace by a combination of colors; e. One or more adjustable knob for dimming light intensity that enable user to liken his or her perception of light in both good and neuropathic eye; f. One or more light sensors to sense the light in the enclosed space for each eye; g. One light control system to change wavelength/color of LED lights; h. Means for alternating illuminating LEDs; i. Means for independently dimming LED lights; j. Means for independently quantifying the light intensity in each enclosed space; k. Means for changing LEDs' light wavelength; l. One timing system that control pupil stimulation including a control algorithm in the processor and the associated circuitry; m. One light control system to set and dim the LED lights for BST and CDT including a control algorithm in the processor and the associated circuitry; n. One or more processors for controlling the methods and algorithms; o. A transmitter system to transmit data either wired or wireless; p. The required power can be provided through an adaptor or through a battery installed in the portable device; q. An analyzer for processing the recorded data and performing image processing algorithms to detect one or more eye structures such as pupil, iris, blood vessels and sclera and/or perform bio-measurements; r. A storage device for storing the results of analyzer and cameras' recordings; s. A control system to enable the tests using the operator's command (the implemented software) including a control algorithm in the processor, user interface on the monitoring device, and the associated circuitry.

The light control system and the means for alternating illuminating LEDs further include: a. a system clock, b. power regulators that derive the appropriate LED, c. circuits to turn LEDs on and off. The adjustable knobs for dimming or changing wavelength further include one or more potentiometers as well as the associated circuitries for each LED. The light sensors include one or more photocells as well as the associated circuitries for each enclosed space.

Each eye will be placed in an enclosed space such that no light will be coming inside from the surrounding environment or from the space associated with the contralateral eye. This will avoid the accommodation and convergence of pupils which may affect the accuracy of tests resulting in false positives. As each enclosed space is completely dark or completely bright, there is no fixation point for the pupils and there will be no accommodation of pupils to reduce the accuracy or increase the chance of false positive.

Dimming of LEDs is used for brightness saturation test (BST) and color discrimination test (CDT).

The device 100 according to the first embodiment can be used for automatic procedure of a swinging-flashlight test comprising: a. A set of driving circuitries and control algorithms for changing the light spectrum of LEDs such that wide-spectrum LEDs generate white light or using white LEDs instead; b. A timing system that control pupil stimulation and photography according to standard protocol of Marcus Gunn Examination; c. Turning on infrared LED lights and illuminating both eyes; d. Turning on white LEDs or the wide-spectrum LEDs, which are generating white light, of the first eye for 2-3 seconds; e. Recording both eyes images and transferring them to the storage device and the processors in real time; f. Turning on the white LEDs or the wide-spectrum LEDs, which are generating white light, of the second eye for 2-3 seconds, while Turning off the white LEDs or the wide spectrum LEDs, which are generating white light, of the first eye, simultaneously; g. Recording both eyes images and transferring them to the storage device and the processors; h. Processing the recorded results and comparing direct and indirect response of pupil and determining pupil size and shape changes; i. Storing the recorded results and the processing results in the storage device; j. Demonstrating all the results in the monitoring device.

The device 100 according to the first embodiment can be used for an automatic procedure of BST comprising: a. Identifying the healthy and abnormal eye by performing the above noted test, or using any other method; b. A set of driving circuitries and control algorithms for changing the light spectrum of LEDs such that wide-spectrum LEDs generate white light or using white LEDs instead; c. A set of control knobs in the monitoring device and associated methods and circuitries to dim the white lights generated by LEDs mentioned in (b); d. Turning on infrared LED lights and illuminating both eyes; e. Turning on the white LEDs or the wide-spectrum LEDs, which are generating white light, for the abnormal eye; f. Setting the white light intensity with the user entered values by means of (c) or pre-saved values; g. Concurrently and continuously taking images from both eyes and transmitting them to the recording device, and the processor in real-time; h. Measuring the size of pupil (diameter, area, or other measures of interest) of the abnormal eye; i. Turning off all LED lights except than IR LEDs; j. Turning on the white LEDs or the wide-spectrum LEDs, which are generating white light, in the enclosed space that is associated with the healthy eye with the user entered values by means of (c) or pre-saved values; k. Concurrently and continuously taking images from both eyes and transmitting them to the recording device, and the processor in real-time; l. Processing the recorded results and determining pupil size (diameter, area, or other measures of interest) in real-time; m. Measuring and recording light intensity levels continuously in the enclosed space associated with the healthy eye; and dimming the white light's intensity until the size of pupil (diameter, area, or other measures of interest) become the same as the recorded values for the pupil of abnormal eye in the presence of the maximum possible value light intensity, or user entered values by means of (c) or pre-saved values; n. Demonstrating the recorded and processed results which includes but is not limited to the difference in light intensity of two enclosed spaces when pupils of both eyes have the same size (diameter, area, or other measures of interest) in the monitoring device in real-time.

The device 100 according to the first embodiment can also be used for a manual procedure of BST comprising: a. A set of driving circuitries and control algorithms for changing the light spectrum of LEDs such that wide-spectrum LEDs generate White light or using white LEDs instead; b. A set of control knobs in the monitoring device, and/or knobs on each side of the portable device (wherein said means (e) in claim 1) and associated methods and circuitries to dim the white lights generated by LEDs mentioned in (a); c. Turning on infrared LED lights and illuminating both eyes; d. Turning on the white LEDs or the wide-spectrum LEDs, which are generating white light, in the enclosed space that is associated with one of the patient's eyes, called the first eye; e. Setting the white light intensity with the user entered values by means of (b) or pre-saved values; f. Concurrently and continuously taking images from both eyes and transmitting them to the recording device and the processor in real-time; g. Measuring the size of pupil (diameter, area, or other measures of interest) of the first eye; h. Turning off the red LEDs or the wide-spectrum LEDs, which are generating white light, in the enclosed space associated with the first eye; i. Turning on the white LEDs or the wide-spectrum LEDs, which are generating white light, in the enclosed space that is associated with the contralateral eye (the second eye) with the user entered values by means of (b) or pre-saved values; j. Concurrently and continuously taking images from both eyes and transmitting them to the recording device and the processor in real-time; k. Concurrently and continuously processing the recorded images and determining pupil size (diameter, area, or other measures of interest) in real-time; l. Measuring and recording light intensity levels continuously in the enclosed space associated with the second eye; while the patient or the operator is changing white light intensity in the enclosed space associated with the second eye using the physical knobs on the portable device or the knobs shown on the monitoring device's screen, respectively; until the patient perceived values of white light in both eyes becomes the same; m. Demonstrating the recorded and processed results which includes but is not limited to the difference in the white light intensity of two enclosed spaces when the perception of the white light intensity by both patient's eyes is the same.

The device 100 according to the first embodiment can also be used for automatic procedure of CDT comprising: a. A set of driving circuitries and control algorithms for changing the light spectrum of LEDs such that wide-spectrum LEDs generate red light or using red LEDs instead; b. A set of control knobs in the monitoring device and associated methods and circuitries to dim the Red lights generated by LEDs mentioned in (a); c. Turning on infrared LED lights and illuminating both eyes; d. Turning on the red LEDs or the wide-spectrum LEDs, which are generating red light, for one of the patient's eyes, called the first eye; e. Setting the red light intensity with the user entered values by means of (b) or pre-saved values; f. Concurrently and continuously taking images from both eyes and transmitting them to the recording device, and the processor in real-time; g. Measuring the size of pupil (diameter, area, or other measures of interest) of the first eye; h. Turning off the red LEDs or the wide-spectrum LEDs which are generating red light; i. Turning on red LEDs or the wide-spectrum LEDs, which are generating red light, in the enclosed space that is associated with the contralateral eye (the second eye) with the user entered values by means of (b) or pre-saved values; j. Concurrently and continuously taking images from both eyes and transmitting them to the recording device, and the processor in real-time; k. Processing the recorded results and determining pupil size (diameter, area, or other measures of interest) in real-time; l. Measuring and recording light intensity levels continuously in the enclosed space associated with the second eye; and dimming the red light's intensity until the size of pupil (diameter, area, or other measures of interest) become the same as the recorded values for the pupil of first eye in the presence of the maximum possible value light intensity, or user entered values by means of (b) or pre-saved values; m. Demonstrating the recorded and processed results which includes but is not limited to the difference in light intensity of two enclosed spaces when pupils of both eyes have the same size (diameter, area, or other measures of interest) in the monitoring device in real-time.

The device 100 according to the first embodiment can also be used for a manual procedure of CDT comprising: a. A set of driving circuitries and control algorithms for changing the light spectrum of LEDs such that wide-spectrum LEDs generate red light or using red LEDs instead; b. A set of control knobs in the monitoring device, and/or knobs on each side of the portable device (wherein said means (e) in claim 1) and associated methods and circuitries to dim the Red lights generated by LEDs mentioned in (a); c. Turning on infrared LED lights and illuminating both eyes; d. Turning on the red LEDs or the wide-spectrum LEDs, which are generating red light, in the enclosed space that is associated with one of the patient's eyes, called the first eye; e. Setting the red light intensity with the user entered values by means of (b) or pre-saved values; f. Concurrently and continuously taking images from both eyes and transmitting them to the recording device and the processor in real-time; g. Measuring the size of pupil (diameter, area, or other measures of interest) of the first eye; h. Turning off the red LEDs or the wide-spectrum LEDs, which are generating red light, in the enclosed space associated with the first eye; i. Turning on the red LEDs or the wide-spectrum LEDs, which are generating red light, in the enclosed space that is associated with the contralateral eye (the second eye) with the user entered values by means of (b) or pre-saved values; j. Concurrently and continuously taking images from both eyes and transmitting them to the recording device and the processor in real-time; k. Concurrently and continuously processing the recorded images and determining pupil size (diameter, area, or other measures of interest) in real-time; l. Measuring and recording light intensity levels continuously in the enclosed space associated with the second eye; while the patient or the operator is changing red light intensity in the enclosed space associated with the second eye using the physical knobs on the portable device or the knobs shown on the monitoring device's screen, respectively; until the patient perceived values of red light in both eyes becomes the same; m. Demonstrating the recorded and processed results which includes but is not limited to the difference in the red light intensity of two enclosed spaces when the perception of the red light intensity by both patient's eyes is the same.

The device 100 according to the first embodiment can also be used for an automatic procedure of color blindness test comprising: a. A set of control knobs in the monitoring device and associated methods and circuitries to perform the test in automatic or manual mode; b. A set of control knobs in the monitoring device for changing the color of LED lights manually; c. A set of driving circuitries and control algorithms for changing the light spectrum of wide-spectrum LEDs. d. Turning on infrared LED lights and illuminating both eyes; e. Turning on the wide-range LED lights for both eyes; f. Setting the starting wavelength of wide-range LEDs' light with the minimum/maximum possible value or with user entered values by means of (b) or pre-saved values; g. Sweeping the light wavelength from the minimum (if starting wavelength of (f) is set to the minimum value) or maximum (if starting wavelength of (f) is set to the maximum value) set wavelength to the maximum (if starting wavelength of (f) is set to the minimum value)) or minimum (if starting wavelength of (f) is set to the maximum value) possible set wavelength with predefined wavelength step size and time interval using the automatic mode or manually by means of (b); h. Measuring and recording light intensity levels continuously in the enclosed space associated with both eyes separately; i. Concurrently and continuously taking images from both eyes and transmitting them to the recording device, and the processor in real-time; j. Measuring the size of pupil (diameter, area, or other measures of interest) of both eyes; k. Demonstrating the recorded and processed results which includes but is not limited to a report which shows the pupil size (diameter, area, or other measures of interest) in response to each color stimulus.

The device 100 according to the first embodiment can also be used for a manual procedure of color blindness test comprising: a. A set of control knobs in the monitoring device and associated methods and circuitries to perform the test in automatic or manual mode; b. A set of control knobs in the monitoring device for changing the color of LED lights manually; c. A set of driving circuitries and control algorithms for changing the light spectrum of wide-spectrum LEDs; d. Turning on infrared LED lights and illuminating both eyes; e. Turning on the the wide-range-LED lights for both eyes with a predefined wavelength; f. Changing the wavelength of lights generated by wide range LEDs by means of (b); g. Measuring and recording light intensity levels continuously in the enclosed space associated with both eyes separately; h. Concurrently and continuously taking images from both eyes and transmitting them to the recording device, and the processor in real-time; i. Measuring the size of pupil (diameter, area, or other measures of interest) of both eyes; j. Demonstrating the recorded and processed results which includes but is not limited to a report which shows pupil size (diameter, area, or other measures of interest) in response to each color stimulus.

In some instances, the imaging devices is typically one or more cameras with or without lenses that photographs both eyes to detect one or more eye structures including but not limited to pupil, iris, and sclera. In this instance, the testing device may comprise: a. A software, implemented on a hand-held monitoring device (including but not limited to tablets, mobile phones, hand-held computers) or on a desktop computer, that lets operator take patient's eyes' images; b. Transmit images to a recording device and the processor in real-time (or with delay) wirelessly or wired; c. An image processing algorithm is being used to analyze the captured images to detect abnormalities. The abnormalities can include (but not limited to) nevus in the iris, inflammation in the iris (hypopyon), blood in the iris (hyphema), and the size, number, and pattern of blood vessels on sclera; d. A comparison algorithm to compare the current and previously recorded images from the same patient and the results of the analysis mentioned in (c), in different instances of time to follow up the progression of each disease and the effect of treatments and surgery.

Disclosed systems and methods may utilize a combination of light sources, sensors, imaging devices, and control algorithms for carrying out assessments of the images captured from eyes of the patient to let a doctor diagnose, compare, and follow up Marcus-Gunn pupil, color blindness, and other eye abnormalities.

The operation of the first embodiment will now be described in further detail with further reference to the figures. FIG. 1 is a schematic functional block diagram of one implementation of an example of the testing device 100 in which the testing device is a pupillary response evaluation system, directed to assessing ophthalmologic biomarkers for assessment of afferent eye defects and color blindness according to one or more aspects of this disclosure. Pupillary response evaluation system may include: a pupillary scanner 117, and a control system 128 that may optionally be embedded in the pupillary scanner 117. The pupillary scanning device 117 may include a scanning system that may be configured to expose eyes of a patient 40 to various light stimuli and concurrently capture images of the eyes in order to record pupillary responses of both eyes as will be described in more detail later in this disclosure. The recorded pupillary responses by the scanning system 117 may be sent as image data via a communication pathway to the control system 128. The control system 128 may include a memory 50 and a processor 52. The memory 50 may include one or more algorithms 54 and the image data 56 received from the scanning system 117. The algorithms 54 may include, but are not limited to executable instructions for performing different ocular assessment procedures (e.g., swinging flashlight test, BST, CDT, and color blindness test, etc.) and evaluation algorithms (e.g., image processing algorithms, etc.) for evaluating the image data 56 obtained from the ocular assessment procedures. The algorithms 54, when executed by the processor 106, may either cause the control system 102 to operate the scanning system 104 for performing different ocular assessment procedures, and/or may cause the control system 102 to evaluate the image data 108 obtained from different ocular assessment procedures for automatically formulating a diagnosis or helping a doctor to formulate a diagnosis, as will be described in more detail later in this disclosure.

In an aspect, pupillary response evaluation system 100 may further include an external computer system 130 that may be operatively connected to the pupillary scanning device 117 and may be configured for allowing a user (i.e., doctor) to monitor the evaluation results produced by the control system 128 and formulate a diagnosis. In an implementation, the external computer system 130 may be operatively connected to the pupillary scanning device 117 using an online connection, for example a secured cloud platform. According to other implementations, the external computer system 130 may be operatively connected to the pupillary scanning device 117 using a physical connection, a local network or the like.

In another implementation (not shown in FIG. 1), the control system 128 may be embedded within the external computer system 130 and it may be operatively connected to the pupillary scanning device 117.

FIG. 2A illustrates a perspective view of an exemplary implementation of the pupillary response scanning device 100. FIG. 2B shows a top view of one exemplary implementation of the pupillary response scanning device 100 configured to provide an example implementation of the FIG. 1 pupillary scanning device 101. FIG. 2C shows a front view of the exemplary pupillary response scanning device 100.

Referring to FIGS. 2A-2C, pupillary response scanning device 100 may include a housing 102 that may provide two isolated eye enclosures 116 for eyes 42 of a test subject (i.e., patient 40) such that each eye may be kept isolated from the other eye and the environment. In an aspect, the housing 102 may be fixed in front of a patient's eyes such that eye enclosures 116 may be pressed against the patient's eye orbitals to prevent light emitted from sources other than the device 100 itself from entering the intended subject's eyes. Eye enclosures 116 may be separated and isolated from one another by a separator 114, such that each eye 42 may be kept in full isolation from the other eye, i.e., no light may be sensed by a contralateral eye when light sources are emitting light to either eyes.

At least two imaging devices (e.g., cameras) 118 are included within eye enclosures 116. Imaging devices 118 may be configured for capturing images of the eyes 42 of the patient during the eye assessment procedures. According to some implementations, each eye enclosure may include at least one imaging device.

With continuing reference to FIGS. 2B and 2C, pupillary response scanning device 100 may include a number of infrared light sources 122, for example infrared light emitting diodes (IR-LEDs) capable of emitting infrared light. Infrared light sources 112 may be arranged on the periphery of imaging devices 118 and directed towards the eyes 42. According to some implementations, at least one infrared light source may be positioned in each eye enclosure that may be directed towards the eye enclosed in that enclosure. Light beams emitted from each infrared light source may shine in each eye and reflect back towards each imaging device. According to an implementation, there may be infrared light sources dedicated to each eye, for example, infrared light sources 122 within each eye space 116 are dedicated to the respective eye 42 associated with that eye space 116.

In an aspect, each eye enclosure 116 may include at least one visible light source 124, such as a light-emitting diode (LED) that may be configured for streaming a wide range of visible light. According to some implementations, visible light source 124 may produce visible light of various intensities and wavelengths. For example, visible light source 124 may be a wide-spectrum LED capable of streaming a wide range of visible light. According to other implementations, visible light source may be a number of colored light sources 126, such as colored LEDs, each streaming a color (e.g., white, red, etc.). In an implementation, visible light sources 124, 126 may be placed in each eye enclosure 116 on the side of each eye 42, such that the light rays emitted from each visible light source 124, 126 may be parallel to the surface of each eye 42.

According to some implementations, each eye enclosure 116 may include at least one light sensor 134 that may be configured for sensing intensity and frequency of light at each moment inside each eye enclosure 116. Light sensors 134 may be place in front of each eye on the periphery of each imaging device and at either sides of each eye space across from the primary visible light sources 124, 126 respectively. Sensors 134 may be configured for converting the amount of light intensity and wavelength in each eye enclosure 116 to electrical signals. The electrical signals may be utilized in a light intensity control mechanism that may be configured to control and adjust the intensity and frequency of light that is generated by each visible light source 124, 126. According to some implementations, a known algorithm, for example, pulse width modulation (PWM) may be utilized for controlling the intensity, wavelength, and frequency of the light generated by visible light sources 124, 126.

Referring to FIG. 2B, the pupillary response scanning device 100 may further include a control system 128 and a power unit 58. The control system 128 may be configured to provide an example implementation of the FIG. 1 control system.

In an aspect, the control system 128 may be configured to manipulate the infrared light sources 122 and the visible light sources 124, 126. As used herein, manipulating means turning the infrared light sources 122 and the visible light sources 124, 126 on and off and changing the intensity and wavelength of the visible light sources 124, 126 based on the executable instructions stored in the memory of the control system 128. Moreover, the control system 128 may be configured to manipulate the imaging devices 118 causing the imaging devices to capture the images of eyes 42 based on the executable instructions stored in the memory of the control system 128.

In some implementations, control system 128 may be operatively coupled to light sensors 134, for purposes that may include, but are not limited to calculating the desired intensity and frequency of the light that may need to be generated by each visible light source 124, 126 inside each eye enclosure 116. The electrical signals sent by light sensors 134 may be fed back to a control algorithm stored on memory 50 (labeled in FIG. 1) to modify the parameters that are being used to generate PWM signals to control the intensity, wavelength, and frequency of the light generated by visible light sources 124, 126.

According to an implementation, power unit 58 may be included in pupillary response scanning device 100 and it may include optional batteries or in another implementation the power may be provided from an external source via a wired connection.

With further reference to FIGS. 2A-2C, according to an implementation, pupillary response scanning device 100 may be designed with a portable goggle-shaped configuration that may be strapped around the eyes of the subject. Consequently, pupillary response scanning device 100 may further include an adjustable strap 104.

Referring to FIGS. 2A and 2B, according to some implementations, pupillary response scanning device 100 may further include adjustment knobs 132 that may be configured for manually adjusting the light intensity of visible light sources 124, 126 in each eye enclosure 116, independently.

In an aspect, the pupillary response scanning device 100 may be configured for evaluating the reactivity of pupil in each eye 42 to both direct and consensual light application. As used herein, direct light application means scanning the pupillary response in the same eye to which light stimulus is being applied by the visible light source 124, 126. Consensual light application means scanning the pupillary response in the eye opposite to that which is receiving light stimulus from the visible light source 124, 126.

### EXAMPLE 1

### Swinging Flashlight Test

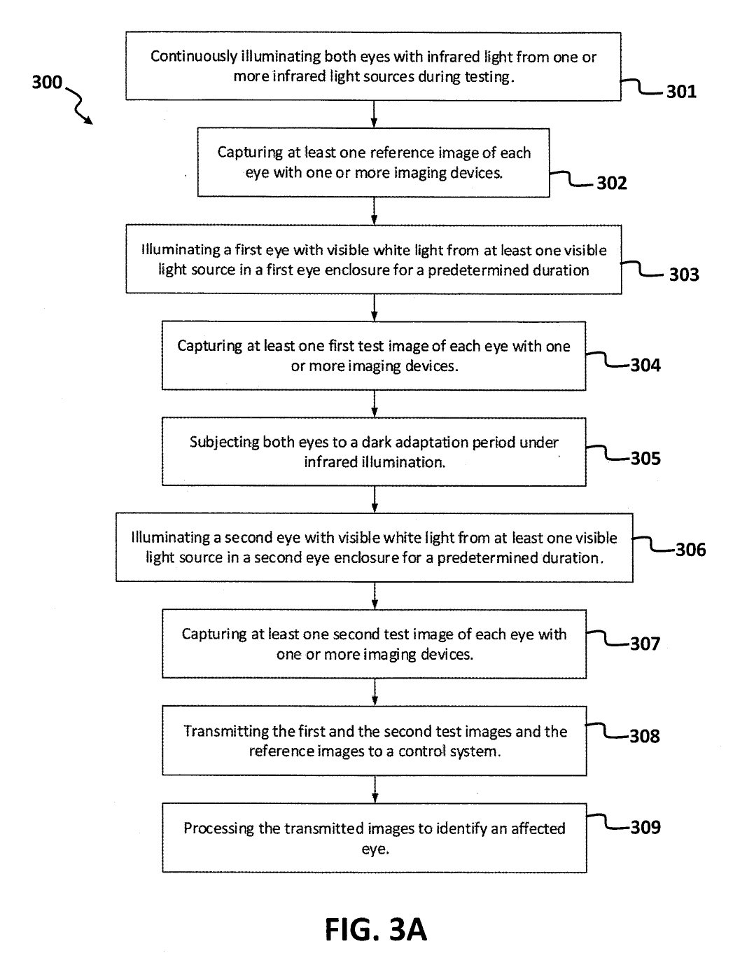

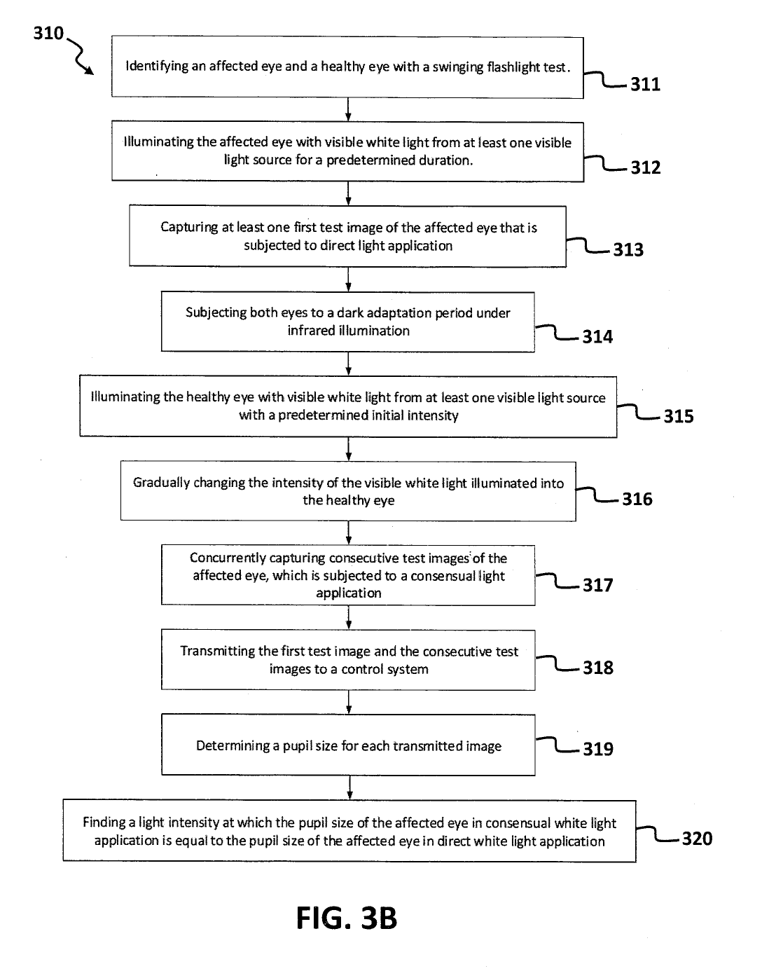

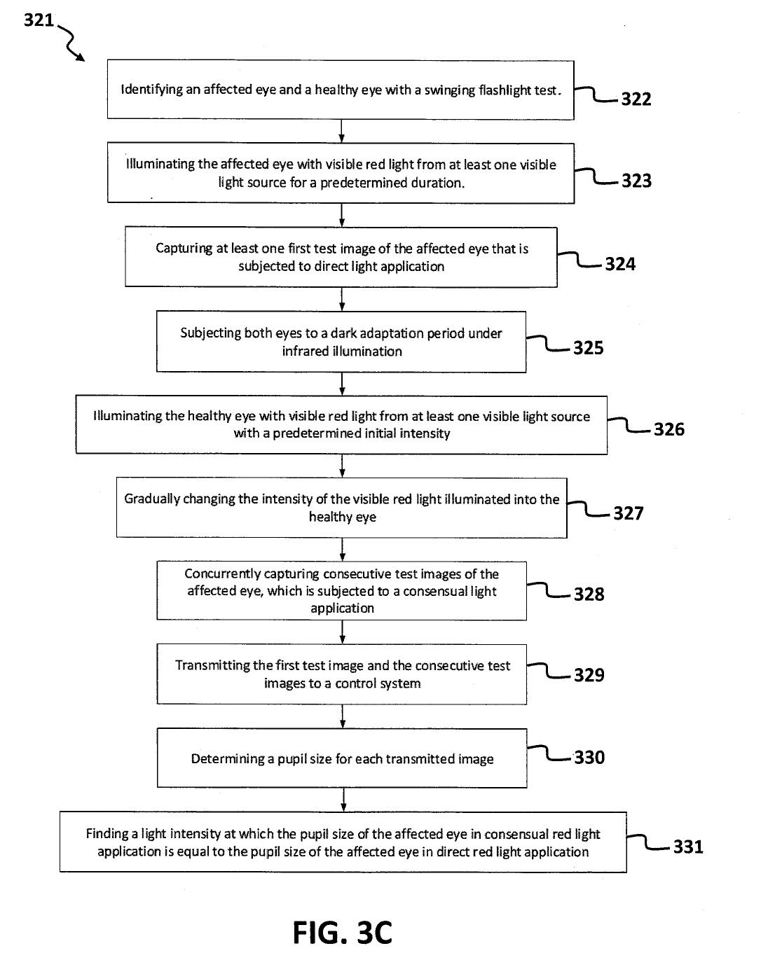

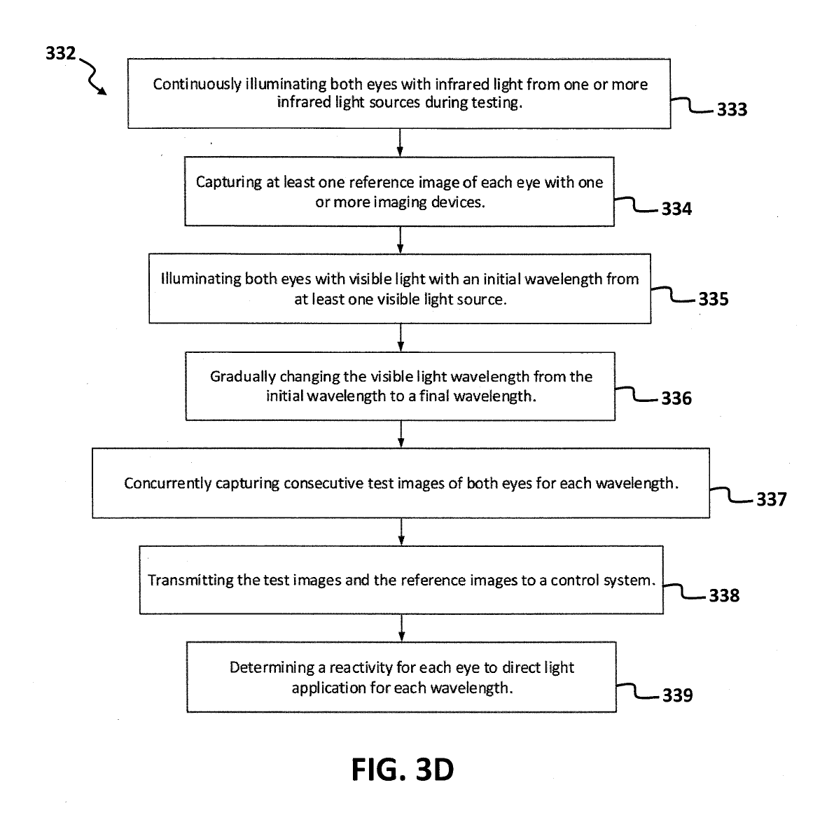

In an aspect, pupillary response scanning device 100 may be configured for performing a swinging flashlight test. FIG. 3A is a logical flow diagram, illustrating one flow 300 of example operations within a swinging flashlight test procedure performed using pupillary response scanning device 100 of the present disclosure. Operations in the flow 300 may include: continuously illuminating both eyes with infrared light from one or more infrared light sources during testing (step 301); capturing at least one reference image of each eye with one or more imaging devices (step 302); illuminating a first eye with visible white light from at least one visible light source in a first eye enclosure for a predetermined duration (step 303); capturing at least one first test image of each eye with one or more imaging devices (step 304); subjecting both eyes to a dark adaptation period under infrared illumination (step 305); illuminating a second eye with visible white light from at least one visible light source in a second eye enclosure for a predetermined duration (step 306); capturing at least one second test image of each eye with one or more imaging devices (step 307); transmitting the first and the second test images and the reference images to a control system (step 308); and processing the transmitted images to identify an affected eye (step 309).

Referring to FIGS. 1 and 3A, operations in the flow 300 may be stored on the memory 105 as executable instructions that once executed by the processor 52 may allow the control system 128 to operate the pupillary response scanning device 100 to perform a swinging flashlight test. As an illustration, referring to FIGS. 2B, 2C, and 3A, example operations of the flow 300 may be performed by the pupillary response scanning device 100 as follows. Referring to 301, infrared light sources 122 in eye enclosures 116 may be turned on in order to continuously illuminate eyes 42.

Moving on to 302, at least one reference image of each eye 42 may be captured with the imaging devices 118 respectively.

Moving on to 303, the visible light source 124, 126 in a first eye enclosure 116 may be turned on in order to illuminate the respective eye 42 with visible white light for an arbitrary and predetermined duration. In some implementations, the predetermined duration may be for example 2 to 3 seconds. Referring to 304, at this point at least one first test image of both eyes 42 may be captured with imaging devices 118. After that, referring to 305, visible light source 124, 126 in one eye enclosure may be turned off in order to subject both eyes 42 to a dark adaptation period under infrared illumination from infrared light sources 122 for a predetermined duration. In some implementations, the dark adaptation period may be a period of 5 seconds.

Moving on to 306, the visible light source 124, 126 in the second eye enclosure 116 may be turned on in order to illuminate the respective eye 42 with visible white light for a predetermined duration. Referring to 307, at this point at least one second test image of both eyes 42 may be captured with imaging devices 118.

With continuing reference to FIGS. 2B, 2C, and 3A, moving on to 308, the reference images captured in 302, the first test images captured in 304, and the second test images captured in 307 may be transmitted to the control system 128. Moving on to 309, referring to FIG. 1, the transmitted images may be stored on the memory 50 as the image data 56. The processor 52 may execute an image processing algorithm that may be stored as executable instructions on the memory 50 to cause the control system 128 to perform operations in order to identify an affected eye. Such operations, may include for example, processing the image data 56 to determine pupillary response measurements, such as pupil reaction/redilation latency, pupil reaction duration, pupil reaction rate, maximal pupil area change, percentage of maximal pupil area change, rebound percentage during redilation. The pupillary response measurements may be carried out by calculating the size of the patient's pupils based on the transmitted images, calculating the amount of constriction in each pupil under both direct and consensual light application by comparing the first and the second test images to the reference images. Accordingly, by comparing the pupillary response of the first eye 42 to pupillary response of the second eye 42 in the image data, the eye with a diminished pupillary response may be identified as the affected or the abnormal eye.

### EXAMPLE 2

### Brightness Saturation Test (BST)

In an aspect, pupillary response scanning device 200 may be configured for performing a BST.

FIG. 3B is a logical flow diagram, illustrating one flow 310 of example operations within a BST procedure performed using pupillary response scanning device 100 of the present disclosure. Operations in the flow 310 may include: identifying an affected eye and a healthy eye with a swinging flashlight test (step 311) that may be performed by exemplary operations of the flow 300 of FIG. 3A; illuminating the affected eye with visible white light from at least one visible light source for a predetermined duration (step 312); capturing at least one first test image of the affected eye that is subjected to direct light application (step 313); subjecting both eyes to a dark adaptation period under infrared illumination (step 314); illuminating the healthy eye with visible white light from at least one visible light source with a predetermined initial intensity (step 315); Gradually changing the intensity of the visible white light illuminated into the healthy eye (step 316); concurrently capturing consecutive test images of the affected eye, which is subjected to a consensual light application (step 317); transmitting the first test image and the consecutive test images to a control system (step 318); determining a pupil size for each transmitted image (step 319); and finding a light intensity at which the pupil size of the affected eye in consensual white light application is equal to the pupil size of the affected eye in direct white light application (step 320).

Referring to FIGS. 1 and 3B, operations in the flow 310 may be stored on the memory 105 as executable instructions that once executed by the processor 52 may allow the control system 128 to operate the pupillary response scanning device 100 to perform a BST.

As an illustration, referring to FIGS. 2B, 2C, and 3B, example operations of the flow 310 may be performed by the pupillary response scanning device 100 as follows. Referring to 311, the affected eye and the healthy eye may be identified by a swinging flashlight test performed by the pupillary response scanning device 100 with a flow of operations as was described in detail in connection with example 1. In this example, a first one of the eyes 42 may be considered to be, for example, the affected eye. Moving on to 312, visible light source 124, 126 in the associated one of the eye enclosures 116 may be turned on in order to illuminate the affected eye with visible white light for an arbitrary and predetermined duration. Referring to 313, at this point at least one first test image of the affected eye may be captured with the associated imaging device 118. After that, referring to 314, visible light source 124, 126 in the associated eye enclosure 116 may be turned off in order to subject both eyes 42 to a dark adaptation period under infrared illumination from infrared light sources 122. Moving on to 315, the visible light source 124, 126 in the other one of the eye enclosures 116 may be turned on in order to illuminate the healthy eye 42 with visible white light with a predetermined initial white light intensity.

Referring to 316, the intensity of the visible white light may be gradually changed by the control system 128 that may receive feedback from light sensors 134. In an implementation, visible white light with a maximum light intensity may be illuminated into the healthy eye and then the light intensity may be reduced from this maximum initial value to lower intensities in a stepwise manner, alternatively, visible white light with a minimum light intensity may be illuminated into the healthy eye and then the light intensity may be increased from this minimum initial value to higher intensities in a stepwise manner. Referring to 317, concurrently consecutive test images may be captured with imaging device 118 from the affected eye while the light intensity of the light source associated with the other eye is being gradually changed in the eye enclosure of the other eye.

Moving on to 318, the first test image and the consecutive test images may be transferred to the control system 128. Referring to FIG. 1, the transmitted images may be stored on the memory 50 as the image data 56. The processor 52 may execute an algorithm that may be stored as executable instructions on the memory 50 to cause the control system 128 to perform operations in order to find a light intensity at which the pupil size of the affected eye in consensual white light application that may be measured by processing is equal to the pupil size of the affected eye in direct white light application. Such operations may include: measuring the pupil size of the affected eye by processing the first test images captured from the affected eye under a direct light application; measuring a pupil size of the affected eye for each consecutive image captured from the affected eye under a consensual light application with different light intensities; and comparing the measured pupil sizes to find a light intensity at which the pupil size of the affected eye in consensual white light application is equal to the pupil size of the affected eye in direct white light application. In an aspect, the difference between the intensities of the direct white light application and the intensity of the consensual light application for which the pupil size of the affected eye is equal to that of the affected eye under direct light application may be utilized by pupillary response scanning device 100 to automatically formulate a quantitative diagnosis or alternatively referring to FIG. 1, the results produced by device 100 may be transmitted to the external computer system 130 and a doctor at the external computer system 130 may utilize the results to formulate a quantitative diagnosis.

### EXAMPLE 3

### Color Discrimination Test (CDT)

In an aspect, pupillary response scanning device 100 may be configured for performing a CDT. FIG. 3C is a logical flow diagram, illustrating one flow 321 of example operations within a CDT procedure performed using pupillary response scanning device 100 of the present disclosure. Operations in the flow 321 may include: identifying an affected eye and a healthy eye with a swinging flashlight test (step 322) that may be performed by exemplary operations of the flow 100 of FIG. 3A; illuminating the affected eye with visible red light from at least one visible light source for a predetermined duration (step 323); capturing at least one first test image of the affected eye that is subjected to direct light application (step 324); subjecting both eyes to a dark adaptation period under infrared illumination (step 325); illuminating the healthy eye with visible red light from at least one visible light source with a predetermined initial intensity (step 326); gradually changing the intensity of the visible red light illuminated into the healthy eye (step 327); concurrently capturing consecutive test images of the affected eye, which is subjected to a consensual red light application (step 328); transmitting the first test image and the consecutive test images to a control system (step 329); determining a pupil size for each transmitted image (step 330); and finding a red light intensity at which the pupil size of the affected eye in consensual red light application is equal to the pupil size of the affected eye in direct red light application (step 331).

Referring to FIGS. 1 and 3C, operations in the flow 321 may be stored on the memory 50 as executable instructions that once executed by the processor 52 may allow the control system 128 to operate the pupillary response scanning device 100 to perform a CDT.

As an illustration, referring to FIGS. 2B, 2C, and 3C, example operations of the flow 321 may be performed by the pupillary response scanning device 100 as follows. Referring to 322, the affected eye and the healthy eye may be identified by a swinging flashlight test performed by the pupillary response scanning device 100 with a flow of operations as was described in detail in connection with example 1. In this example, a first one of the eyes may be considered to be, for example, the affected eye. Moving on to 323, visible light source 124, 126 in eye enclosure associated with the affected eye may be turned on in order to illuminate the affected eye with visible red light for an arbitrary and predetermined duration. Referring to 324, at this point at least one first test image of the affected eye may be captured with the associated imaging device 118. After that, referring to 325, visible light source 124, 126 in the eye enclosure associated with the affected eye may be turned off in order to subject both eyes 42 to a dark adaptation period under infrared illumination from infrared light sources 122. Moving on to 326, visible light source 124, 126 in a second one of the eye enclosures may be turned on in order to illuminate the healthy eye with visible red light with a predetermined initial red light intensity. Referring to 327, the intensity of the visible red light may be gradually changed by the control system 128 that may receive feedback from light sensors 134. In an implementation, visible red light with a maximum light intensity may be illuminated into the healthy eye and then the light intensity may be reduced from this maximum initial value to lower intensities in a stepwise manner, alternatively, visible red light with a minimum light intensity may be illuminated into the healthy eye and then the light intensity may be increased from this minimum initial value to higher intensities in a stepwise manner. Referring to 328, concurrently consecutive test images may be captured with the imaging device associated with the affected eye while the light intensity of the light source is gradually decreased in the eye enclosure of the healthy eye.

Moving on to 329, the first test image and the consecutive test images may be transferred to the control system 128. Referring to FIG. 1, the transmitted images may be stored on the memory 50 as the image data 56. The processor 52 may execute an algorithm that may be stored as executable instructions on the memory 50 to cause the control system 128 to perform operations in order to find a red light intensity at which the pupil size of the affected eye in consensual red light application is equal to the pupil size of the affected eye in direct red light application. Such operations may include: measuring the pupil size of the affected eye by processing the first test images captured from the affected eye under a direct red light application; measuring a pupil size of the affected eye for each consecutive image captured from the affected eye under a consensual red light application with different light intensities; and comparing the measured pupil sizes to find a red light intensity at which the pupil size of the affected eye in consensual red light application is equal to the pupil size of the affected eye in direct red light application. In an aspect, the difference between the intensities of the direct red light application and the intensity of the consensual red light application for which the pupil size of the affected eye is equal to that of the affected eye under direct red light application may be utilized by pupillary response scanning device 100 to automatically formulate a quantitative diagnosis or alternatively referring to FIG. 1, the results produced by device 100 may be transmitted to the external computer system 130 and a doctor at the external computer system 130 may utilize the results to formulate a quantitative diagnosis.

### EXAMPLE 4

### Color Blindness Test

In an aspect, pupillary response scanning device 100 may be configured for performing a color blindness test. FIG. 3D is a logical flow diagram, illustrating one flow 332 of example operations within a color blindness test procedure performed using pupillary response scanning device 100 of the present disclosure. Operations in the flow 332 may include: continuously illuminating both eyes with infrared light from one or more infrared light sources during testing (step 333); capturing at least one reference image of each eye with one or more imaging devices (step 334); illuminating both eyes with visible light with an initial wavelength from at least one visible light source (step 335); gradually changing the visible light wavelength from the initial wavelength to a final wavelength (step 336); concurrently capturing consecutive test images of both eyes for each wavelength (step 337); transmitting the test images and the reference images to a control system (step 338); determining a reactivity for each eye to direct light application for each wavelength (step 339); and determining the sensitivity of each eye to different visible light wavelengths based on the determined reactivity for each eye (step 340).

Referring to FIGS. 1 and 3D, operations in the flow 332 may be stored on the memory 50 as executable instructions that once executed by the processor 52 may allow the control system 128 to operate the pupillary response scanning device 100 to perform a color blindness test.

As an illustration, referring to FIGS. 2B, 2C, and 3D, example operations of the flow 332 may be performed by the pupillary response scanning device 100 as follows. Referring to 333, infrared light sources 122 in a first one of the eye enclosures 116 and infrared light sources 122 in a second one of the eye enclosure 116 may be turned on in order to continuously illuminate both eyes. Moving on to 334, at least one reference image of the first eye may be captured and at least one reference image of the second eye may be captured with the respective imaging devices 118. Moving on to 335, visible light sources 124, 126 in both eye enclosures may be turned on in order to illuminate both eyes with visible light with a predetermined initial wavelength. Referring to 336, the wavelength of the visible light may be gradually changed by the control system 128 that may receive feedback from light sensors 134. In an implementation, visible light with a maximum wavelength may be illuminated into both eyes and then the wavelength may be reduced from this maximum initial value to lower wavelengths in a stepwise manner, alternatively, visible light with a minimum wavelength may be illuminated into both eyes and then the wavelength may be increased from this minimum initial value to higher wavelengths in a stepwise manner. Referring to 337, concurrently consecutive test images may be captured with imaging devices from both eyes while the wavelength of the light sources associated with both eyes are being gradually changed in the eye enclosures.

Moving on to 338, the reference images captured in 334, and the consecutive test images captured in 337 for each wavelength may be transmitted to the control system 128. Moving on to 339, referring to FIG. 1 the transmitted images may be stored on the memory 50 as the image data 56. The processor 52 may execute an image processing algorithm that may be stored as executable instructions on the memory 50 to cause the control system 128 to perform operations in order to carry out pupillary response measurements, such as determining the reactivity of each eye to direct light application for each wavelength. Based on the reactivity of each eye to direct visible light application with different wavelengths, the sensitivity of each eye to different visible light wavelengths that corresponds to different visible light colors may be determined. In an aspect, the device 100 may formulate a diagnosis automatically based on the obtained results or alternatively the device 100 may only report the reactivity of each eye to each color stimulus and a user (i.e., doctor) at the external computer system 130 may use the reports transmitted by the device 100 to formulate a diagnosis.

Referring to FIGS. 2A-2C, in an aspect, the BST and CDT procedures may be performed manually utilizing the pupillary response scanning device 100 of the present disclosure. The light intensities in the BST and CDT procedures may be changed using adjustment knobs 132 manually by the patient or an operator and instead of using an algorithm to automatically finding a light intensity for which the pupil size of the affected eye in consensual white or red light applications is equal to the pupil size of the affected eye in direct white or red light applications, the operator may ask the patient to change the intensity using the adjustment knobs 132 and find the right intensity based on their judgment, i.e., patient's perceived intensities of consensual white or red light applications becomes the same as patient's perceived intensities of direct white or red light applications.

### EXAMPLE 5

### Eye Abnormality Detection Test

In an aspect, pupillary response scanning device 100 may be configured for performing eye abnormality detection tests. To this end, images may be captured from the whole surface of each eye including pupil, iris, and sclera. The captured images may then be sent to a control system that may employ an image processing algorithm to process the captured images for detecting, analyzing, and tracking any abnormalities in short and long term. The abnormalities may include, but are not limited to, nevus in the iris, inflammation in the iris (i.e., hypopyon), blood in the iris (i.e., hyphemia), surgery marks, and the size, number, and pattern of blood vessels on sclera.

Referring to FIGS. 1, 2B, and 2C, as an illustration, example operations of eye abnormality detection test may be performed by the pupillary response scanning device 100 as follows: imaging devices 118 may capture images of the entire surface of eyes 42. The captured images may then be transmitted to the control system 128 to be stored on the memory 50 as the image data 56. The memory 50 may include image processing algorithms that once executed by the processor 52, may cause the control system 128 to detect abnormalities in eyes 42. The control system 128 may be configured to store the test results of a patient in the memory 50 for future references and it may be configured to track the abnormalities in eyes of a patient based on the recorded test results on the memory 50.

According to some implementations, the predetermined duration for visible light stimulation of each eye may be 2 to 3 seconds. In other implementations, the dark adaptation period may be a period of for example 5 seconds.

Turning now more particularly to the embodiment of FIGS. 4 through 17, in this instance the computer device 130 may be integrally supported on the computer device so as to be carried on the head of the user together with the remaining components of the testing device 100. The display screen 131 of the computer device 130 in this instance is typically located on the rear side of the housing for outputting information away from the front side of the housing which is engaged on a person to be tested. Captured eye images can be displayed on the display screen 131 mounted at the rear side of the portable housing, as shown in FIG. 4, in real-time, together with calculated results and relevant criteria for assessing certain conditions of the patient being overlaid with the eye images or displayed separately.

Within each eye space, the main visible light source 124, 126 is typically provided at a location rearwardly of the front side of the housing within the respective eye space for directing visible light forwardly onto the eyes of the person so that the light is directed along a path which is substantially parallel to a line of sight of the respective eye. Alternatively, the visible light emitting diode 126 within each eye space may comprise a plurality of different colour LEDs which collectively provide a full/wide spectrum of white light. Typically, the main light emitting diode is provided within each eye space at a location which is spaced laterally outwardly in alignment along a common horizontal axis with the camera so as to be spaced rearwardly of the front side of the housing to direct light forwardly onto the respective eye of the person.

In the embodiment of FIGS. 4 through 17, the visible light source may further include an auxiliary array of light emitting diodes 136 and an additional convergence light emitting diode 138 within each eye space, in addition to the main light emitting diode 124 described generally above. The array of LEDs 136 includes a plurality of individual LEDs which are positioned in a generally annular relationship about the respective camera such that the LEDs are evenly spaced apart along a generally rectangular perimeter path. Within the rectangular perimeter path, individual LEDs are provided at each of the four corners of the path and at evenly spaced positions along each side so as to include LEDs which are spaced apart horizontally from one another along a horizontal top row and a horizontal bottom row as well as including LEDs which are spaced apart vertically from one another along an inner vertical row and an outer vertical row relative to the camera. A central LED within the two vertical rows within each eye space define respective left and right light emitting diodes of the eye space at diametrically opposed locations relative to the camera so as to be aligned along a common horizontal row with the camera therebetween. The left and right light emitting diodes of each array of diodes 136 are used individually in some eye tests described in further detail below.

The two convergence light emitting diodes 138 are located in the two eye spaces respectively such that each convergence LED is located to be offset laterally inwardly from the respective camera towards the other eye space. The two convergence LEDs are thus suitably positioned so as to be aligned with one another at a common focal point of the two eyes of the user when both are activated.

The main visible light sources 124, 126 for generating visible light in the embodiment of FIGS. 4 through 17 comprise a single wide spectrum LED 124, or multiple different color LEDs 126 to provide white light similarly to the first embodiment, but the visible light sources 124, 126 in this instance are supported within the cavity to project direct light rearwardly onto respective eyes of the patient. The visible light sources remain positioned within each eye space 116 laterally offset outwardly in relation to the respective camera 118.

In the embodiment of FIGS. 4 through 17, each of the light emitting diodes generating visible light within the eye spaces respectively, including the light sources 124, 126, 136, and 138, is provided with a covering frame portion 140 covering the LED at a location forward relative to the LED so as to be located between the LED and the eyes of a person being tested. The covering frame portion 140 includes a single aperture 142 therein in alignment with each LED in which the aperture has a diameter which is smaller than a corresponding dimension of the light emitting surface of the respective LED. In this manner, the light emitted from the LED is limited by the aperture 142 of the covering frame portion to be a single point or dot corresponding to the size and shape of the aperture.

In further embodiments, any of the features of the first embodiment of FIGS. 1 through 3 may be incorporated into the device according to the embodiment of FIGS. 4 through 17. More particularly the device of FIGS. 4 through 17 may be modified to include manual inputs 132 located externally on the housing for use by the person being tested or an operator to controllably vary an operating characteristic of any one of the tests, for example intensity or wavelength of light being generated. Furthermore, the device according to the embodiment of FIGS. 4 through 17 may be modified to include light sensors 134 which convert light signals within the respective eye space to electrical signals fed back to the controller for modifying various operational characteristics of the controller, or to merely record relevant lighting conditions within each eye space during a particular test being performed such that the lighting conditions can be recorded as additional data attached to the results of the prescribed test for validating the test results at a later date.

Turning now to the operation of the second embodiment of FIGS. 4 to 17, the testing device 100 in this instance is used as a DUI or DWI screening device. The device contains a goggle-shaped frame to be located on the head and in front of the eyes, an optional strap to secure the frame on the eye, an optional touch screen on the front so the user can control the operation and view the results, and an optional handle so the user can fix the device position in front of the head of another person.

FIG. 5 schematically illustrates an embodiment of the computer device 130 provided as a touch-screen hand-held device that can optionally being used to control the operation of the DUI or DWI screening device and record and view its test results instead of or in addition to the integrated computer within the portable housing which is controlled through the rear display screen 131. The hand-held device will be connected to the DUI or DWI screening device in FIG. 1, using a wireless communication. The wireless communication can be, but is not limited to, WiFi or Bluetooth communication. The software on the hand-held device will let the user to choose which tests to run. It will send commands through the wireless communication to the DUI or DWI screening device, and will receive back the camera recordings in real time. The software will perform an image processing algorithm to analyze the results of the tests, and the results will be demonstrated to the user on the hand-held device. A simple schematic of software as displayed on the screen of the hand-held device 130 is shown in FIG. 2, but the actual software user interface may vary form the shown schematic. The hand held device may display captured images 80 in real time, together with input selections 82 which can be selected by interaction with the touch screen device upon which they are displayed, as well as the calculated results 84 of the tests.

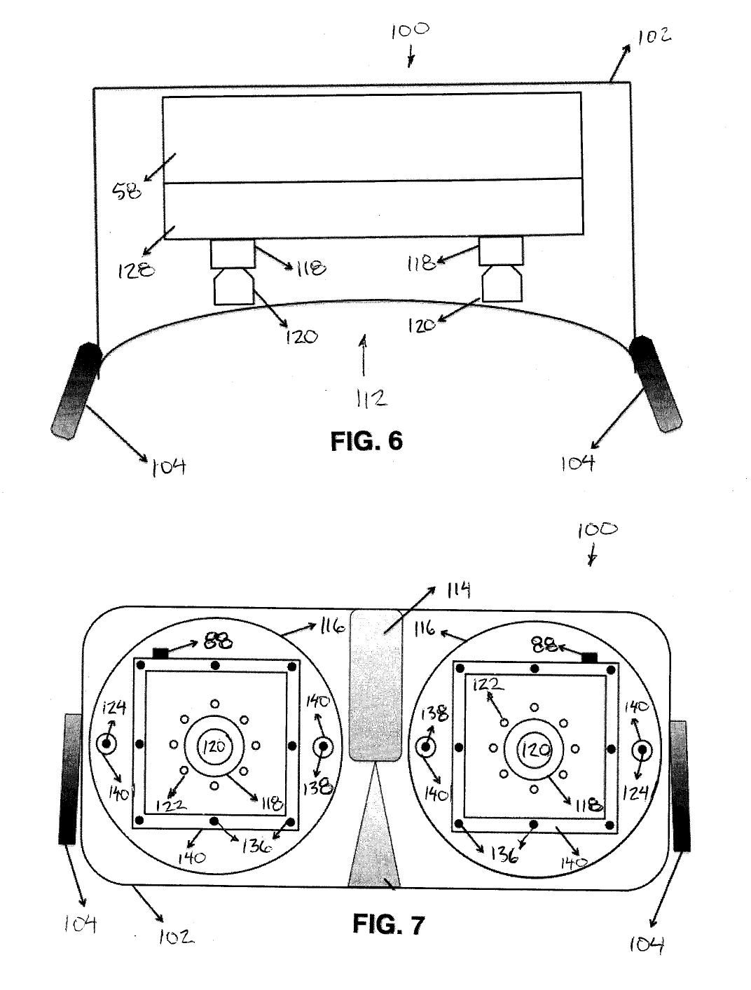

FIG. 6 demonstrates the top view of the goggle-shaped DUI or DWI screening device. The batteries 58 are used to power up the device, and the electronics control circuit board 128 controls the operation of the device. The electronic control circuit board also contains a wireless communication module to transmit data between the electronic circuit board 128 and the software on the hand-held device 130. The cameras and all the lights are controlled via the electronic control circuit board based on the test being run.

FIG. 7 demonstrates the front view of the goggle-shaped DUI or DWI screening device. The front view contains two eyepieces 116 and a designed space for placing nose. The eyepieces are isolated from each other using a separator 114. In the middle of each eyepiece there is a camera 118 and lens system 120 to record the eye and its movements. Around the camera, one or more Infrared LED(s) 122 are placed to enable the camera to record videos and take photos in the dark.

Around the Infrared LED(s) 122, a small rectangular-shaped frame 140 which covers eight or more white LEDs 136 which are evenly installed on the LED-driver printed circuit board. The distribution of the white LEDs 136 is such that each side of the rectangle will have the same and odd number of white LEDs. The rectangular-shaped frame has small holes in the place of white LEDs, so the light of the LEDs will be pin-shaped.

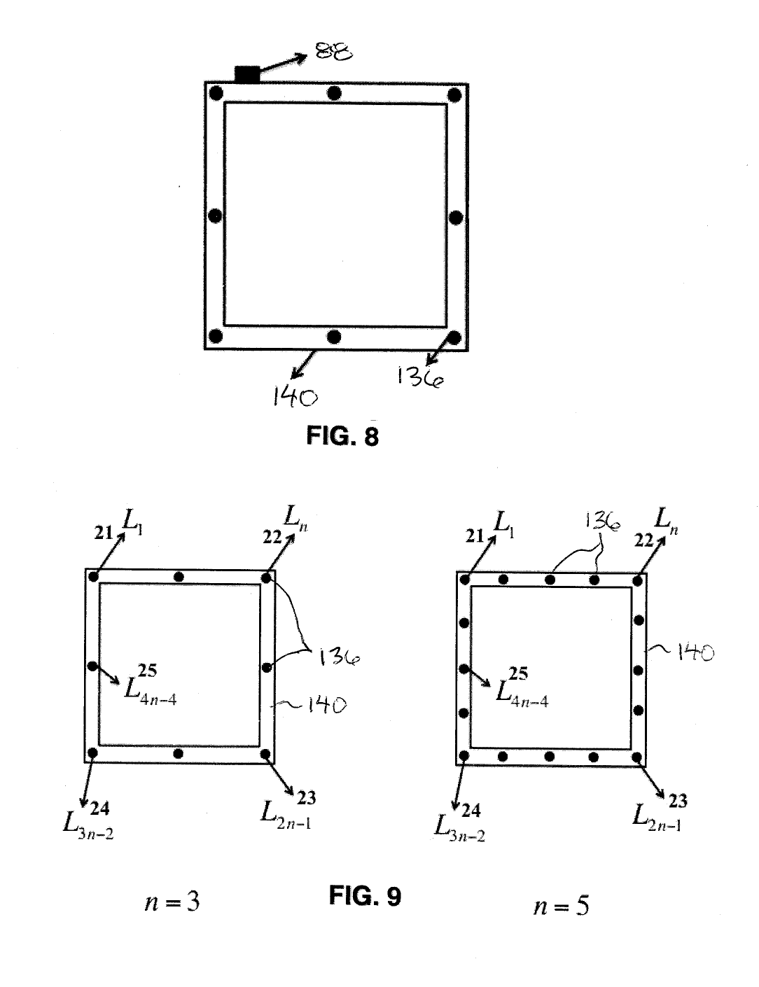

FIG. 8 shows the LED-driver printed circuit board, which contains LED driver circuits for turning individual white LEDs on and off, and a connector terminal 88, so that power and control signals can be transferred to the LED-driver printed circuit board from the electronic control circuit board. The LED-driver printed circuit board, will include all the required electronics to drive the white LEDs.

The white LEDs can vary in numbers. FIG. 9 demonstrates two of the possible distributions but in general the design is not limited to the number of LEDs shown. Also, FIG. 9 shows the naming convention for each white LED. L1 shows the white LED on the top left corner. Ln shows the white LED on the top right corner. Based on this there should be always n white LEDs on each side of the LED-driver printed circuit board, which the value of n can be any odd number larger than 3. Also, in this naming convention, the white LED in the bottom right corner is named L2n-1, the LED in the bottom left corner is named L3n-2, and the last LED in the loop (one below the top left corner) is named L4n-4. Therefore, the total number of white LEDs will be (4n-4).

Two or more white LEDs are located at each side of each eye, covered by a frame with a small hole to make a pin-shaped light. These LEDs are also being controlled individually depending on the test being performed.

The presented DUI or DWI screening device along with its software, can perform at least 8 different tests related to DUI or DWI screening with eye examination. The user will have access to run individual tests and view their results. However, the results of individual tests may be integrated with specific integration algorithms to result in better accuracy of DUI or DWI screening.

In all the tests performed by the DUI or DWI screening device, the cameras, and the infrared LEDs 122 are always turned on before the start of the test. Also, the camera controlling software is set such that it automatically records videos of eye movement and will stream the video in real time to the software installed on the hand-held device. The image processing algorithms will run automatically on the recorded videos and will demonstrate the results related to the test being performed.

The integration algorithms are outside of the scope of this patent. The details of the individual tests are as follows:

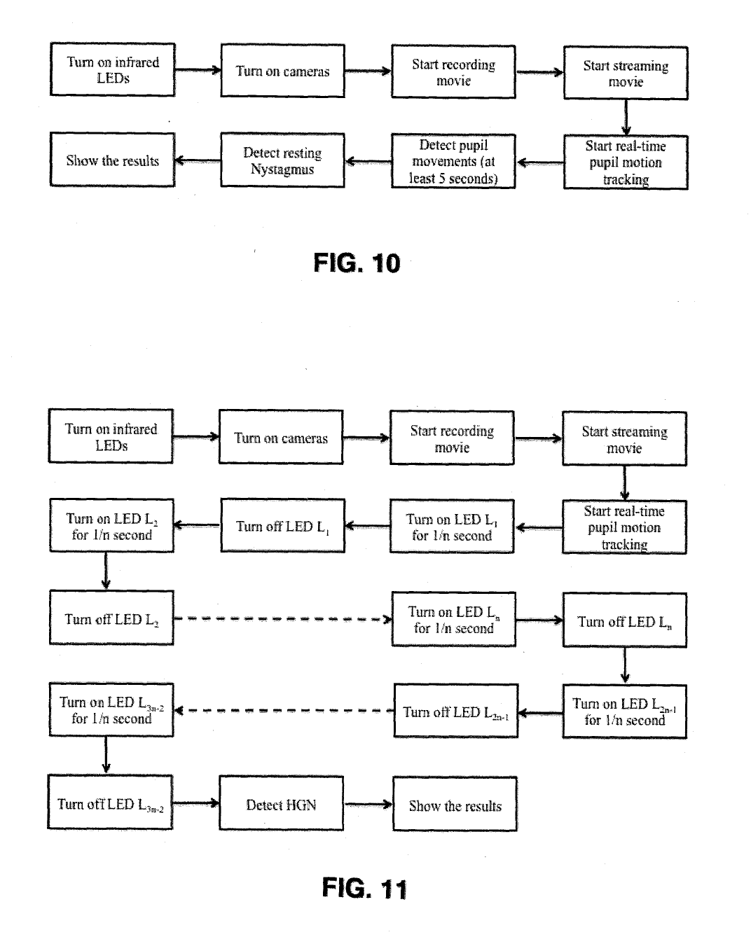

Test 1: Resting Nystagmus. This test is being done by keeping the eyepieces in dark and recording videos of the movement of the eye. The steps of this test are shown in FIG. 10. Test 1 is designed based on the fact that in some cases of the person being under influence of drugs or alcohol, at resting position of eye, the pupils will have sudden and jerky movements. Utilizing the image processing algorithms for this test, the position of the pupil is tracked, so that any movements will be recorded and will be used to determine that if the person has the resting Nystagmus condition or not.

Test 2: Horizontal Gaze Nystagmus (HGN). This test is being done by keeping the eyepieces in dark initially, and recording videos of the movement of the eye. Then, lights of the array 136 will be illuminated as follows. The white LED L1 will be turned on for 1/n seconds. Then the second white LED L2 will be turned on for 1/n seconds. This will repeat until at the end of the 1-second period of time, white LED Ln will be turned on. Overall during the last 1 second white LEDs on the top will be turned on and off one-by-one starting from the left. During the next step the bottom white LEDs 136 will be turned on and off one by one starting from the right with the time periods of 1/n seconds. During both steps the camera 118 will record videos of the pupils movement while the infrared LEDs 122 are on at all times. The steps of this test are shown in FIG. 11. This test is designed based on the fact that in some cases of the person being under influence of drugs or alcohol, the pupil cannot follow the objects horizontally and will have sudden or jerky movements. Utilizing the image processing algorithms for this test, the position of the pupil is tracked, so that any movements will be recorded and will be used to determine that if the person has the HGN condition or not. The steps of this steps being shown in FIG. 11 may be performed for both eyes simultaneously or one-by-one individually.

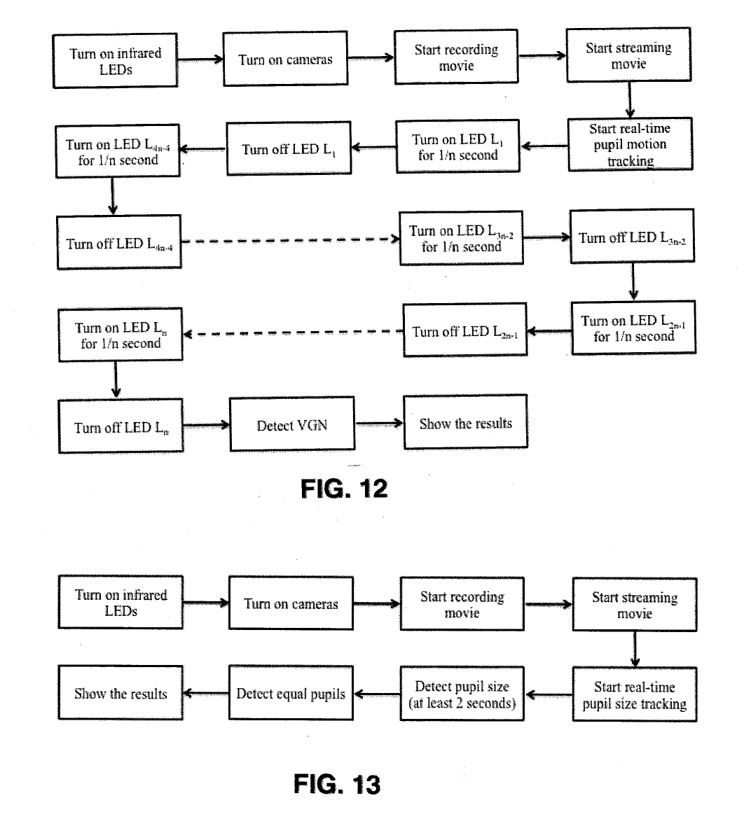

Test 3: Vertical Gaze Nystagmus (VGN). This test is being done by keeping the eyepieces in dark initially, and recording videos of the movement of the eye. Then, the array of LEDs 136 will be illuminated as follows. The white LED L1 will be turned on for 1/n seconds. Then the second white LED L4n-4 will be turned on for 1/n seconds. This will repeat until at the end of the 1-second period of time, white LED L3n-2 will be turned on. Overall during the last 1 second white LEDs on the left will be turned on and off one-by-one starting from the top. During the next step the right white LEDs will be turned on and off one by one starting from the bottom with the time periods of 1/n seconds. During both steps the camera will record videos of the pupils movement while the infrared LEDs 122 are on at all times. The steps of this test are shown in FIG. 12. This test is designed based on the fact that in some cases of the person being under influence of drugs or alcohol, the pupil cannot follow the objects vertically and will have sudden or jerky movements. Utilizing the image processing algorithms for this test, the position of the pupil is tracked, so that any movements will be recorded and will be used to determine that if the person has the VGN condition or not. The steps of this steps being shown in FIG. 12 may be performed for both eyes simultaneously or one-by-one individually.

Test 4: Equal pupils. This test is being done by keeping the eyepieces in dark and recording videos of the movement of the eye. The steps of this test are shown in FIG. 13. Test 4 is designed based on the fact that in some cases of the person being under influence of drugs or alcohol, the pupil sizes of eyes are different. Utilizing the image processing algorithms for this test, the size of the pupils are measured, so that any difference will be recorded and will be used to determine that if the person has the not-equal pupils condition or not.

Test 5: Lack of smooth pursuit. This test is being done by keeping the eyepieces in dark initially, and recording videos of the movement of the eye. Then, the white LED L1 will be turned on for 1/n seconds. Then the second white LED L2 will be turned on for 1/n seconds. This will repeat until at the end of the (4n-4)/n-second period of time, white LED L4n-4 will be turned on. Overall during the last (4n-4)/n seconds, all white LEDs will be turned on and off one-by-one starting from the top left. The steps of this test are shown in FIG. 14. This test is designed based on the fact that in some cases of the person being under influence of drugs or alcohol, the pupil cannot smoothly pursuit the moving objects and will have non-smooth movements. Utilizing the image processing algorithms for this test, the position of the pupil is tracked, so that any movements will be recorded and will be used to determine that if the person has the Lack of Smooth Pursuit condition or not. The steps of this steps being shown in FIG. 14 may be performed for both eyes simultaneously or one-by-one individually.

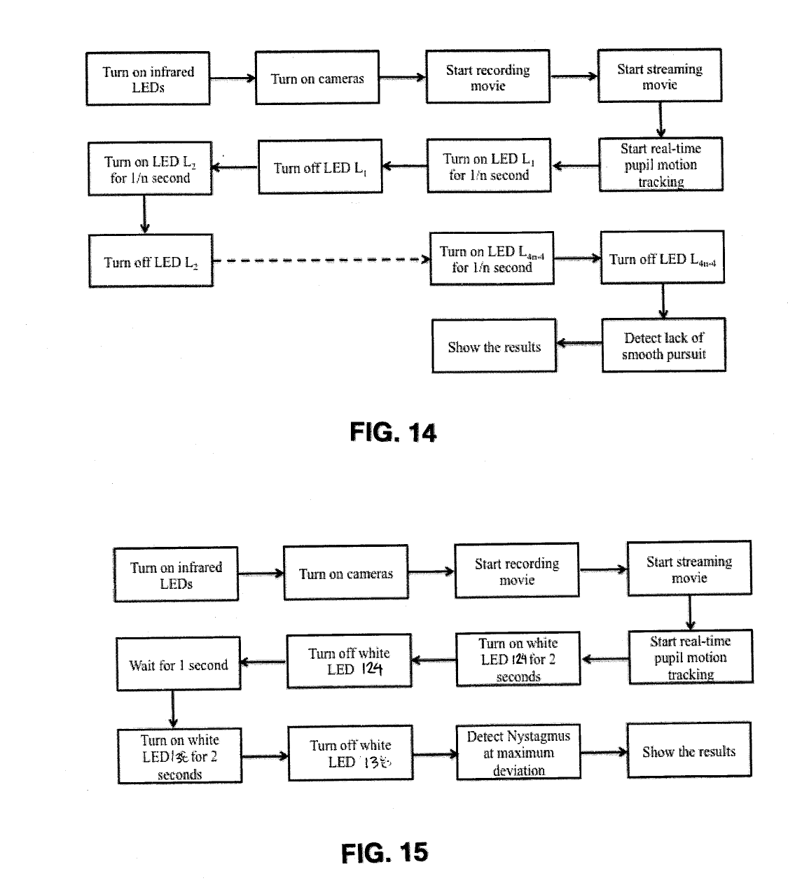

Test 6: Nystagmus at maximum deviation. This test is being done by keeping the eyepieces in dark initially, and recording videos of the movement of the eye. Then, the white LED 124 will be turned on for 2 seconds and will be turned off after that. After 1 second of resting, the white LED 138 will be turned on for 2 seconds and will be turned off after that. The steps of this test are shown in FIG. 15. Test 6 is designed based on the fact that in some cases of the person being under influence of drugs or alcohol, at maximum deviation of eyes, the pupils will have sudden and jerky movements. Utilizing the image processing algorithms for this test, the position of the pupil is tracked, so that any movements will be recorded and will be used to determine that if the person has the Nystagmus at Maximum Deviation condition or not. The steps of this steps being shown in FIG. 15 will be performed for each eye individually.

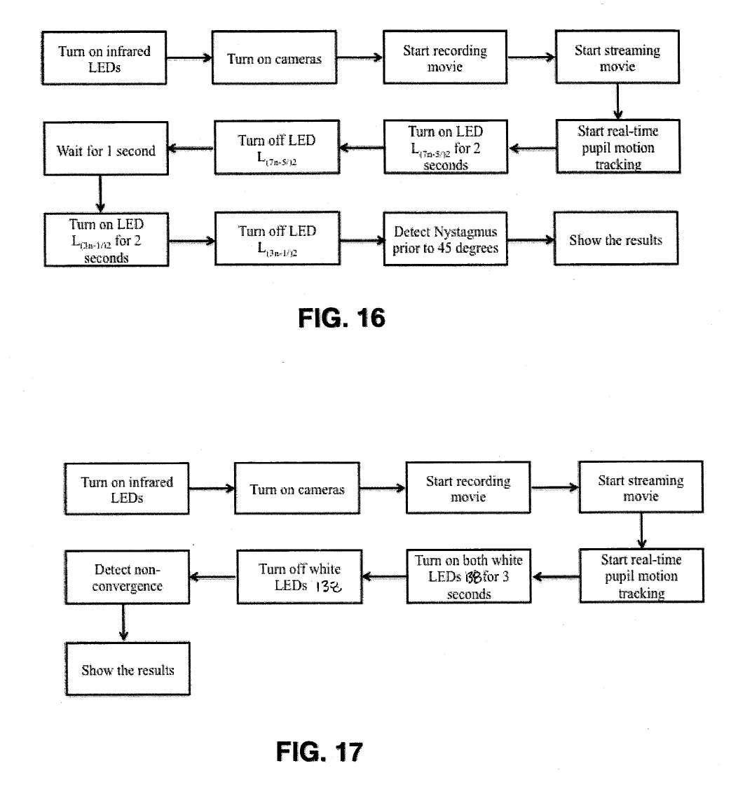

Test 7: Nystagmus prior to 45 degrees. This test is being done by keeping the eyepieces in dark initially, and recording videos of the movement of the eye. Then, the white LED in the middle of the left side, L(7n-5)/2, will be turned on for 2 seconds and will be turned off after that. After 1 second of resting, the white LED in the middle of the right side, L(3n-1)/2, will be turned on for 2 seconds and will be turned off after that. The steps of this test are shown in FIG. 16. Test 7 is designed based on the fact that in some cases of the person being under influence of drugs or alcohol, at 45-degrees deviation of eyes, the pupils will have sudden and jerky movements. Utilizing the image processing algorithms for this test, the position of the pupil is tracked, so that any movements will be recorded and will be used to determine that if the person has the Nystagmus Prior to 45 Degrees condition or not. The steps of this steps being shown in FIG. 16 will be performed for each eye individually.

Test 8: Non-convergence. This test is being done by keeping the eyepieces in dark initially, and recording videos of the movement of the eye. Then, the white LEDs 138 will be turned on for 3 seconds (for both eyes simultaneously) and then turned off. The steps of this test are shown in

FIG. 17. Test 8 is designed based on the fact that in some cases of the person being under influence of drugs or alcohol, the eyes will have the issue of not being able to converge to the same point. Utilizing the image processing algorithms for this test, the position of the pupil is tracked, so that any movements will be recorded and will be used to determine that if the person has the Non-convergence condition or not.

In the illustrated embodiment, the testing device for screening driving under influence of drugs or alcohol, or driving while intoxicated with drug or alcohol using eye test, the system comprises: at least one imaging system taking images or video of each eye at a fixed location; at least one infrared light generator for each eye at a fixed location; at least ten individually-controlled white lights 136 for each eye at fixed and predefined locations; two isolated eyepieces to include the imaging system, the infrared light generators and the white lights, while minimizing the effect of outside disturbances such as ambient lighting conditions; a means of wireless communication with a hand-held device; a means of powering the electronics, the lights, and the imaging system; a portable hand-held electronic device in proximity to the device connected to the device using the wireless communication, the portable hand-held electronic device comprising a wireless communication system, and a software to control the performance of the device based on user's commands.

The testing device is typically provided with algorithms for performing eight different eye tests, individually, and showing their results to the user. The algorithms include: steps of performing resting Nystagmus eye test; steps of performing the Horizontal Gaze Nystagmus eye test; steps of performing the Vertical Gaze Nystagmus eye test; steps of performing the Lack of Smooth Pursuit eye test; steps of performing the Equal Pupil eye test; steps of performing the Nystagmus at Maximum Deviation eye test; steps of performing the Nystagmus Prior to 45 Degrees eye test; and steps of performing the Non-convergence eye test.

Turning now to a third embodiment shown in FIG. 18, the testing device 100 in this instance includes all of the features of the first and second embodiments of FIGS. 1 through 17 of the testing device combined. Accordingly, the testing device in this instance is capable of executing any of the above noted tests using features from any of the above noted embodiments.

Since various modifications can be made in my invention as herein above described, and many apparently widely different embodiments of same made, it is intended that all matter contained in the accompanying specification shall be interpreted as illustrative only and not in a limiting sense.

1. An ocular response testing device for testing eyes of a patient to perform a screening test to screen for driving under influence of drugs or alcohol, the testing device comprising:
a portable housing arranged to be supported in proximity to the eyes of the patient, the housing including two eye spaces which are isolated from one another for independent interaction with the eyes of the patient respectively;
an infrared light generator on the housing within each eye space for illuminating the eye spaces respectively with infrared light;
an imaging system on the housing associated with both eye spaces for capturing images of both eyes of the patient when aligned with the two eye spaces for illumination by the infrared light generator respectively;
a visible light source on the housing in association with each eye space for selectively producing visible light within the eye spaces respectively; and
a controller supported on the housing which is operable to illuminate the visible light source within each eye space according to a prescribed pattern and to capture images of both eyes in response to said prescribed pattern according to a prescribed test.

2. to 21. (canceled)

22. The testing device according to claim 1 wherein the testing device is adapted to preform a plurality of screening tests selected from the list consisting of: (i) a resting Nystagmus eye test; (ii) a Horizontal Gaze Nystagmus eye test; (iii) a Vertical Gaze Nystagmus eye test; (iv) a Lack of Smooth Pursuit eye test; (v) an Equal Pupil eye test; (vi) a Nystagmus at Maximum Deviation eye test; (vii) a Nystagmus Prior to 45 Degrees eye test; (viii) a Non-convergence eye test.

23. The testing device according to claim 1 wherein the screening test includes a resting Nystagmus eye test, the testing device further comprising programming instructions for the controller adapted to:
(i) turn on the infrared light generator in each eye space while maintaining the eye spaces in darkness;
(ii) capture images to generate a stream of eye images associated with each eye space;
(iii) concurrently in real-time, identify a pupil in the captured images of each stream of eye images and track a corresponding movement of the pupil for a prescribed duration;
(iv) determine if a resting Nystagmus condition is present in the streams of eye images;
(v) generate a notification through an output of the testing device if the resting Nystagmus condition is determined to be present.

24. The testing device according to claim 1 wherein the screening test includes an Equal Pupil eye test, the testing device further comprising programming instructions for the controller adapted to:
(i) turn on the infrared light generator in each eye space while maintaining the eye spaces in darkness;
(ii) capture images of respective eyes of the patient associated with each eye space;
(iii) identify a pupil in the captured images of each eye space and determine a size of the identified pupil within each eye space;
(iv) compare a difference between the size of the identified pupils of the two eye spaces to an equal pupil criteria to determine if a non-equal pupil condition is present in the eye images; and
(v) generate a notification through an output of the testing device if the non-equal pupil condition is determined to be present.

25. The testing device according to claim 1 wherein the visible light source comprises a sequence of light emitting diodes supported within each eye space in spaced apart relation with one another.

26. The testing device according to claim 25 wherein the sequence of light emitting diodes within each eye space comprise a horizontal row of light emitting diodes.

27. The testing device according to claim 26 wherein the screening test includes a Horizontal Gaze Nystagmus test, the testing device further comprising programming instructions for the controller adapted to:
(i) turn on the infrared light generator in each eye space;
(ii) illuminate the light emitting diodes in sequence with one another along the horizontal row of light emitting diodes;
(iii) capture images to generate a stream of eye images associated with each eye space while the light emitting diodes are sequentially illuminated;
(iv) identify a pupil in the captured images of each stream of eye images and track a corresponding movement of the pupil;
(v) compare the movement from each stream of eye images to a horizontal gaze criteria to determine if a Horizontal Gaze Nystagmus condition is present in the eye images; and
(vi) generate a notification through an output of the testing device if the Horizontal Gaze Nystagmus condition is determined to be present.

28. The testing device according to claim 25 wherein the sequence of light emitting diodes within each eye space comprise a vertical row of light emitting diodes.

29. The testing device according to claim 28 wherein the screening test includes a Vertical Gaze Nystagmus test, the testing device further comprising programming instructions for the controller adapted to:
(i) turn on the infrared light generator in each eye space;
(ii) illuminate the light emitting diodes in sequence with one another along the horizontal row of light emitting diodes;
(iii) capture images to generate a stream of eye images associated with each eye space while the light emitting diodes are sequentially illuminated;
(iv) identify a pupil in the captured images of each stream of eye images and track a corresponding movement of the pupil;
(v) compare the movement from each stream of eye images to a vertical gaze criteria to determine if a Vertical Gaze Nystagmus condition is present in the eye images; and
(vi) generate a notification through an output of the testing device if the Vertical Gaze Nystagmus condition is determined to be present.

30. The testing device according to claim 25 wherein the sequence of light emitting diodes within each eye space follow a generally annular path in which some of the light emitting diodes are horizontally spaced apart from one another and some of the light emitting diodes are vertically spaced apart from one another.

31. The testing device according to claim 30 wherein the screening test includes a Lack of Smooth Pursuit test, the testing device further comprising programming instructions for the controller adapted to:
(i) turn on the infrared light generator in each eye space;
(ii) illuminate the light emitting diodes in sequence with one another along the generally annular path of light emitting diodes;
(iii) capture images to generate a stream of eye images associated with each eye space while the light emitting diodes are sequentially illuminated;
(iv) identify a pupil in the captured images of each stream of eye images and track a corresponding movement of the pupil;
(v) compare the movement from each stream of eye images to a smooth pursuit criteria to determine if a Lack of Smooth Pursuit condition is present in the eye images; and
(vi) generate a notification through an output of the testing device if the Lack of Smooth Pursuit condition is determined to be present.

32. The testing device according to claim 25 wherein each light emitting diode includes a covering frame portion covering the light emitting diode including a pin hole therein such that any light emitted by the light emitting diode is pin-shaped.

33. The testing device according to claim 25 wherein the screening test includes a Nystagmus at maximum deviation test, the testing device further comprising programming instructions for the controller adapted for each eye space to:
(i) turn on the infrared light generator in the eye space;
(ii) activate the visible light source to illuminate the eye space for a first duration;
(iii) inactivate the visible light source such that the eye space is in darkness for a second duration;
(iv) activate the visible light source to illuminate the eye space for a third duration;
(v) capture images to generate a stream of eye images associated with the eye space during the first duration, the second duration and the third duration;
(vi) identify a pupil in the captured images of the stream of eye images for the eye space and track a corresponding movement of the pupil;
(vii) compare the movement from the stream of eye images to a maximum deviation criteria to determine if a Nystagmus at maximum deviation condition is present in the eye images; and
(viii) generate a notification through an output of the testing device if the Nystagmus at maximum deviation condition is determined to be present.

34. The testing device according to claim 33 wherein the visible light source in each eye space comprises a light emitting diode and a covering frame portion covering the light emitting diode including a pin hole therein such that any light emitted by the light emitting diode is pin-shaped.

35. The testing device according to claim 25 wherein the imaging system comprising a camera in each eye space for alignment with the respective eye of the patient and wherein the visible light source comprises a left light emitting diode and a right light emitting diode in each eye space at respective diametrically opposing sides of the camera.

36. The testing device according to claim 35 wherein the screening test includes a Nystagmus prior to 45 Degrees test, the testing device further comprising programming instructions for the controller adapted for each eye space to:
(i) turn on the infrared light generator in the eye space;
(ii) activate the left light emitting diode for a first duration while the right light emitting diode is inactive;
(iii) inactive both left and right light emitting diodes for a second duration;
(iv) activate the right light emitting diode for a third duration while the left light emitting diode is inactive;
(v) capture images to generate a stream of eye images associated with the eye space during the first duration, the second duration and the third duration;
(vi) identify a pupil in the captured images of the stream of eye images for the eye space and track a corresponding movement of the pupil;
(vii) compare the movement from the stream of eye images to a 45 degree criteria to determine if a Nystagmus prior to 45 degrees condition is present in the eye images; and
(viii) generate a notification through an output of the testing device if the Nystagmus prior to 45 degrees condition is determined to be present.

37. The testing device according to claim 35 wherein each of the left and right light emitting diodes includes a covering frame portion covering the light emitting diode and including a pin hole therein such that any light emitted by the light emitting diode is pin-shaped.

38. The testing device according to claim 25 wherein the imaging system comprising a camera in each eye space for alignment with the respective eye of the patient and wherein the visible light source comprises a convergence light emitting diode in each eye space at a location which is laterally inwardly relative to the respective camera such that the two convergence light emitting diodes appear to converge with one another from a perspective of the eyes of the patient.

39. The testing device according to claim 38 wherein the screening test includes a Non-convergence test, the testing device further comprising programming instructions for the controller adapted for each eye space to:
(i) turn on the infrared light generator in the eye space;
(ii) capture images to generate a stream of eye images associated with each eye space;
(iii) simultaneously activate both convergence light emitting diodes for a prescribed duration;
(iv) identify a pupil in the captured images of the stream of eye images for both eye spaces and track a corresponding movement of the pupils;
(v) compare the movement from the stream of eye images to a non-convergence criteria to determine if a Non-convergence condition is present in the eye images; and
(vi) generate a notification through an output of the testing device if the Non-convergence condition is determined to be present.

40. The testing device according to claim 38 wherein each convergence light emitting diode includes a covering frame portion covering the convergence light emitting diode including a pin hole therein such that any light emitted by the convergence light emitting diode is pin-shaped.

41. to 48. (canceled)
